(19) United States
(12) Patent Application Publication
(10) Pub. No.: US 2026/0273227 A1
(43) Pub. Date: Sep. 17, 2026

(54) MEDICAL DEVICE HANDLE

(71) Applicant: Biosense Webster (Israel) Ltd., Yokneam (IL)

(72) Inventors: Sreekanth Reddy Rampa, Irvine, CA (US); Jasson Rodriguez, Irvine, CA (US); Michael Govea, Glendale, CA (US); Wyatt Griffin, Irvine, CA (US); Audrey Vu, Irvine, CA (US)

(21) Appl. No.: 19/435,298

(22) Filed: Dec. 29, 2025

Related U.S. Application Data
(60) Provisional application No. 63/772,448, filed on Mar. 14, 2025.

(51) Int. Cl. A61M 25/01

(52) U.S. Cl. CPC A61M 25/0136; A61M 25/0113; A61M 25/0147; A61M 2205/103

(57) ABSTRACT

The present disclosure provides a medical device handle assembly comprising a handle shell and a bevel gear assembly configured to be inserted into the handle shell. The bevel gear assembly includes a first bevel gear and a second bevel gear in mechanical communication with the first bevel gear. One end of the bevel gear assembly is configured to connect to an outer shaft knob. The second bevel gear includes a mating interface configured to engage with a servo shaft when the handle assembly is positioned on a stabilizer. The stabilizer is configured to control movement of the outer shaft knob via the servo shaft when the handle assembly is positioned on the stabilizer.

### CROSS-REFERENCE TO RELATED APPLICATIONS

This application claims priority to U.S. Provisional Patent Application No. 63/772,448, titled “SYSTEMS AND METHODS FOR IMPROVING CATHETER MANEUVERABILITY”, filed Mar. 14, 2025, which is hereby incorporated by reference in its entirety.

### BACKGROUND

Medical devices have become increasingly sophisticated, offering advanced capabilities for diagnosis and treatment. Handles for these devices play a crucial role in facilitating precise control and manipulation during medical procedures. As procedures become more complex, there is an ongoing effort to enhance the functionality and usability of medical device handles.

Many medical procedures require precise control of catheter tips or other instrument ends. This often involves both rotational and deflection movements to navigate through anatomical structures or perform specific interventions. Traditional handle designs may limit the operator's ability to achieve fine control, particularly when multiple degrees of freedom are needed simultaneously.

Durability and reliability are paramount concerns for medical device handles. These instruments must withstand repeated use and sterilization processes while maintaining consistent performance. Designing mechanisms that are both robust and precise often requires innovative approaches to component integration and sealing.

As medical procedures evolve, there is an increasing demand for handles that can accommodate multiple functions within a compact form factor. This may include controls for deflection, rotation, fluid delivery, or various therapeutic interventions. Balancing these diverse capabilities with ergonomic considerations and ease of use remains an ongoing area of development in medical device design.

The integration of electronic components and sensors into medical device handles offers potential for enhanced functionality and data collection. However, this also introduces complexities related to power management, signal processing, and ensuring compatibility with other medical systems and equipment.

Efforts continue in the medical device industry to develop handle designs that offer improved control, efficiency, and versatility across a range of procedures. Addressing these challenges may lead to advancements that enhance both the capabilities of medical devices and the ability of healthcare professionals to deliver effective patient care.

Improvements are needed.

### SUMMARY

It is to be understood that both the following general description and the following detailed description are exemplary and explanatory only and are not restrictive. Systems and methods for a medical device handle to facilitate automated independent tip rotation and deflection control are described herein.

Disclosed herein is a novel handle. The novel handle is an advanced, intuitive control device designed to enhance the maneuverability and functionality of medical devices, such as catheters, in medical procedures while remaining cost-effective. Unlike traditional manual handles, the novel handle design integrates sensors, haptic feedback, and wireless communication to provide precise control and real time data exchange with the overall system, improving both safety and outcomes at a significantly lower cost.

Disclosed herein are a novel handle and accompanying stabilizer. The novel handle may comprise a handle, such as a catheter hangle, and the accompanying stabilizer may receive the novel handle and facilitate operation of the novel handle while coupled to the novel handle. The novel handle and accompanying stabilizer may comprise an intuitive control device designed to enhance the maneuverability and functionality of medical devices, such as catheters, in medical procedures while remaining cost-effective. Unlike traditional manual handles, the design of the novel handle and accompanying stabilizer may integrate sensors, haptic feedback, and wireless communication to provide precise control and real-time data exchange with an overall imaging system, improving both safety and outcomes at a significantly lower cost. Incorporation of the accompanying stabilizer may allow for fully autonomous manipulation of a medical device without a need for physician intervention, enabling hands-free operation.

Disclosed herein is a medical device controller (e.g., catheter controller, controller). The controller may eliminate a need for a traditional handle, allowing the controller to steer a medical device, such as a catheter, without manual input from a user. The design of the controller features a compact connector mounted on a shaft, such as a catheter shaft, that houses only a deflection mechanism and electronics like an electrically erasable programmable read-only memory (EEPROM), a printed circuit board (PCB), etc. By removing a need for manual catheter manipulation, a user can focus solely on steering a therapeutic device. This simplified design not only streamlines workflow and reduces complexity but also lowers manufacturing costs by shifting control to the controller and minimizing medical device components (catheter components, etc.).

These and other features and advantages are described in greater detail below.

### BRIEF DESCRIPTION OF THE DRAWINGS

Non-limiting and non-exhaustive examples are described with reference to the following figures.

FIGS. 1-13 show an example catheter handle described herein.

FIGS. 14-18 show an example stabilizer described herein.

FIGS. 19-21 show an example catheter controller 300 described herein.

FIG. 22 illustrates a section view of a bevel gear mechanism according to the present disclosure.

FIG. 23 illustrates a sectional view of a handle assembly incorporating a bevel gear mechanism according to the present disclosure.

FIG. 24 illustrates an exploded view of a mounting assembly according to the present disclosure.

FIG. 25 illustrates a section view of a mounting assembly according to the present disclosure.

FIG. 26 illustrates a side view of a mechanical assembly with a bevel gear mechanism and mounting components according to the present disclosure.

FIG. 27 illustrates a side view of a mechanical assembly showing integration of bevel gears and spindle components according to the present disclosure.

FIG. 28 illustrates an exploded view of a handle assembly according to the present disclosure.

FIG. 29 illustrates an exploded view of a handle assembly from a different perspective according to the present disclosure.

FIG. 30 illustrates a section view of a handle assembly showing internal components according to the present disclosure.

FIG. 31 illustrates a sectional view of a handle assembly with various internal components according to the present disclosure.

FIG. 32 illustrates a view of a mounting assembly for a catheter system according to the present disclosure.

FIG. 33 illustrates a perspective view of a mounting assembly for a catheter system according to the present disclosure.

FIG. 34 illustrates multiple views of a motor shaft connection assembly according to the present disclosure.

FIG. 35 illustrates an exploded view of a motor adapter assembly according to the present disclosure.

FIG. 36 illustrates a section view of mating components for a mechanical interface system according to the present disclosure.

FIG. 37 illustrates a section view of a deflection knob insert according to the present disclosure.

FIG. 38 illustrates a section view of a motor adapter mechanism under applied force according to the present disclosure.

FIG. 39 illustrates a section view of a motor adapter assembly according to the present disclosure.

FIG. 40 illustrates a flowchart of a method for assembling a handle with a bevel gear mechanism according to the present disclosure.

FIG. 41 illustrates a flowchart of a method for assembling a handle device according to the present disclosure.

FIG. 42 illustrates a flowchart of a method for connecting a motor adapter to a medical device handle according to the present disclosure.

The accompanying drawings show examples of the disclosure. It is to be understood that the examples shown in the drawings and/or discussed herein are non-exclusive and that there are other examples of how the disclosure may be practiced.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

A traditional catheter is controlled and maneuvered by a physician, requiring additional devices for operation. This means the physician must manage two separate devices, which can become cumbersome. The traditional catheter is manually operated using a handle housing that allows a physician to control the catheter's movement. This manual process often requires switching between multiple devices to achieve the desired positioning. The handle housing is typically large and bulky to accommodate hand placement and grip, resulting in the need for larger packaging, increased table space, higher costs, and substantial user input. The systems and methods described herein may overcome limitations of the traditional catheter.

Disclosed herein is a novel handle. The novel handle is an advanced, intuitive control device designed to enhance the maneuverability and functionality of catheters in medical procedures while remaining cost-effective. Unlike traditional manual handles, the novel handle design integrates sensors, haptic feedback, and wireless communication to provide precise control and real time data exchange with the overall system, improving both safety and outcomes at a significantly lower cost.

The novel handle may comprise electronic deflection controls. Multiple button configurations are possible.

The novel handle may comprise pre-programmed curves for specific anatomy, such as a left atrial appendage (LAA), left atrium (LA), valves, etc.

The novel handle may comprise auto-return to return to a home (initial, etc.) setting. The novel handle may comprise potential parked workflow automation.

The novel handle may facilitate auto scanning of anatomy with location tag. An anatomical location may be tagged for auto scanning.

The novel handle may enable remote control of use with a three-dimension anatomical navigation and mapping system.

The novel handle may comprise programmable limits.

The novel handle may comprise integrated sensors, such as one or more gyroscope, accelerometer, pressure sensor, etc., for fine-tuned control. The novel handle may comprise provide feedback on exact position, angle, force, etc.

The novel handle may comprise haptic feedback to provide tactile feedback to a user.

The novel handle may comprise wireless communication to send real-time data to another device.

The novel handle may comprise customizable controls.

The novel handle may provide automated assistive features with built-in machine learning (ML) (e.g., artificial intelligence (AI)) guidance to suggest or partially control movement based on real-time imaging or data analytics.

The novel handle may comprise built-in safety alerts for excessive force, proximity to critical structures, battery level warnings with emergency override function to switch to manual control, etc.

Disclosed herein are a novel handle and accompanying stabilizer. The novel handle may comprise a catheter handle and the accompanying stabilizer may receive the novel handle and facilitate operation of the novel handle while coupled to the novel handle. The novel handle and accompanying stabilizer may comprise an intuitive control device designed to enhance the maneuverability and functionality of catheters in medical procedures while remaining cost-effective. Unlike traditional manual handles, the design of the novel handle and accompanying stabilizer may integrate sensors, haptic feedback, and wireless communication to provide precise control and real-time data exchange with an overall imaging system, improving both safety and outcomes at a significantly lower cost. Incorporation of the accompanying stabilizer may allow for fully autonomous manipulation of a catheter without a need for physician intervention, enabling hands-free operation.

The novel handle and accompanying stabilizer may allow full catheter control. The accompanying stabilizer may comprise a controller.

The novel handle and accompanying stabilizer may significantly reduce cost for single use a catheter. The novel handle and accompanying stabilizer may allow for removal of equipment and complexity removed from a catheter and placed on and/or in capital equipment (e.g., the accompanying stabilizer).

The novel handle and accompanying stabilizer may improve workflow for a user. The novel handle and accompanying stabilizer may eliminate or reduce a need to manipulate multiple catheters at a time.

The novel handle and accompanying stabilizer may facilitate ultrasound image optimization through closed loop integration with an imaging system.

The novel handle and accompanying stabilizer may use a feedback loop from an imaging system to enable machine learning (ML) (e.g., artificial intelligence (AI)) guidance for real-time imaging and/or data analytics.

The novel handle and accompanying stabilizer may comprise electronic deflection controls. Multiple button configurations are possible.

The novel handle and accompanying stabilizer may comprise pre-programmed curves for specific anatomy, such as a left atrial appendage (LAA), left atrium (LA), valves, etc.

The novel handle and accompanying stabilizer may comprise auto-return to return to a home (initial, etc.) setting. The novel handle and accompanying stabilizer may comprise potential parked workflow automation.

The novel handle and accompanying stabilizer may facilitate auto scanning of anatomy with location tag. An anatomical location may be tagged for auto scanning.

The novel handle and accompanying stabilizer may comprise integrated sensors, such as one or more gyroscope, accelerometer, pressure sensor, etc., for fine-tuned control. The novel handle and accompanying stabilizer may comprise provide feedback on exact position, angle, force, etc.

The novel handle and accompanying stabilizer may comprise haptic feedback to provide tactile feedback to a user.

The novel handle and accompanying stabilizer may comprise wireless communication to send real-time data to another device.

The novel handle and accompanying stabilizer may comprise built-in safety alerts for excessive force, proximity to critical structures, battery level warnings with emergency override function to switch to manual control, etc.

The novel handle and accompanying stabilizer may enable remote operation of the catheter.

Disclosed herein is a catheter controller (e.g., controller). The catheter controller may eliminate a need for a traditional catheter handle, allowing the controller to steer the catheter without manual input from a user. The design of the controller features a compact connector mounted on a catheter shaft that houses only a deflection mechanism and electronics like an electrically erasable programmable read-only memory (EEPROM), a printed circuit board (PCB), etc. By removing a need for manual catheter manipulation, a user can focus solely on steering a therapeutic device. This simplified design not only streamlines workflow and reduces complexity but also lowers manufacturing costs by shifting control to the controller and minimizing catheter components.

The catheter controller may comprise full catheter control using a processor and/or a stabilizer.

The controller may significantly reduce cost for single use a catheter. The controller may allow for removal of equipment and complexity removed from a catheter and placed on and/or in capital equipment (e.g., the controller).

The controller may improve workflow for a user. The controller may eliminate or reduce a need to manipulate multiple catheters at a time.

The controller may facilitate ultrasound image optimization through closed loop integration with an imaging system.

The controller may use a feedback loop from an imaging system to enable machine learning (ML) (e.g., artificial intelligence (AI)) guidance for real-time imaging and/or data analytics.

The controller may comprise electronic deflection controls. Multiple button configurations are possible.

The controller may comprise pre-programmed curves for specific anatomy, such as a left atrial appendage (LAA), left atrium (LA), valves, etc.

The controller may comprise auto-return to return to a home (initial, etc.) setting. The controller may comprise potential parked workflow automation.

The controller may facilitate auto scanning of anatomy with location tag. An anatomical location may be tagged for auto scanning.

The controller may comprise integrated sensors, such as one or more gyroscope, accelerometer, pressure sensor, etc., for fine-tuned control. The controller may comprise provide feedback on exact position, angle, force, etc.

The controller may comprise haptic feedback to provide tactile feedback to a user.

The controller may comprise wireless communication to send real-time data to another device.

The controller may comprise built-in safety alerts for excessive force, proximity to critical structures, battery level warnings with emergency override function to switch to manual control, etc.

The controller may enable remote operation of the catheter.

The present disclosure includes a bevel gear mechanism (e.g., assembly, structure, etc.) that is designed to be inserted into a handle shell. This assembly may comprises bevel gears arranged within a structure, such as a cartridge, which can be removable and/or integrated into the handle shell. The bevel gear assembly enables precise control and rotation functionality.

The modular design may allow for easier assembly, maintenance, and potential replacement of the bevel gear mechanism within the medical device handle. This approach could provide modularity and flexibility in the manufacturing and servicing of the device.

The present disclosure relates to a smart handle with an independent tip rotation mechanism for medical devices. A bevel gear mechanism may be disposed into the handle shell. One end of the bevel gear mechanism (or cartridge comprising the bevel gear mechanism) may connect to an outer shaft knob. The end may comprise one bevel gear. A second bevel gear may have a mating part that interfaces with a servo shaft when placed on a smart handle stabilizer. Servo shaft-driven arrangement described may enable precise and independent tip rotation. The design may facilitate enhanced procedural accuracy and operator control. A safety feature allows slipping when a certain force threshold is exceeded. The present disclosure addresses the need for seamless, precise control in medical procedures. The modular design may facilitate manufacturing, maintenance, and potential replacement of components.

The present disclosure includes a subassembly of a medical device handle containing pull lines and an independent tip rotation mechanism, designed to be pre-assembled as a single unit. The subassembly may be configured to be disposed (e.g., slide, be placed, etc.) easily into a handle shell, simplifying the assembly process. The design may incorporate one or more spindle for deflection control, allowing for precise manipulation of the medical device tip. The pull lines and tip rotation mechanism may be integrated within a single cartridge structure, promoting a compact and efficient design. The subassembly may be removable from the handle shell, facilitating maintenance or replacement of components. The handle assembly may include water-tight seals to enhance durability and maintain functionality. The modular design may streamline manufacturing, improve ease of assembly, and potentially reduce maintenance complexity for medical device handles used in procedures requiring precise control and manipulation.

The present disclosure describes a smart handle with an integrated tip rotation and deflection module for medical devices. The smart handle may comprise a subassembly containing pull lines and an independent tip rotation mechanism, pre-assembled as a single unit. The subassembly may be designed to slide easily into a one-piece handle shell, simplifying assembly. The subassembly may comprise two spindles incorporated: one spindle for controlling left-right deflection, the other spindle for controlling up-down deflection. The subassembly may comprise a cartridge design that allows mounting of bevel gears, enabling independent tip rotation functionality. The integrated design may simplify assembly of pull lines and tip rotation components. The subassembly may comprise water-tight seals durability and functionality. The modular design may facilitate manufacturing, maintenance, and potential replacement of components. The present disclosure addresses the need for efficient assembly and precise control in medical procedures. The smart handle design combines multiple control functions into a compact, modular unit, potentially improving manufacturing efficiency and device functionality for medical applications benefitting from precise tip control and rotation.

The present disclosure comprises a motor adapter system for connecting a medical device handle to a motor. A motor adapter may be configured to be positioned on a motor shaft. A deflection knob insert may be designed to mate with the motor adapter. The present disclosure may comprise alignment components to facilitate connection and prevent misalignment. Alignment components may comprise tapered splines to guide the deflection knob insert onto the motor adapter. Alignment components may comprise an intermediate adapter with teeth that engage with teeth on the motor adapter. Alignment components may comprise a spring element to bias the intermediate adapter away from the motor adapter. Alignment components may comprise splined interfaces with chamfered features for easier alignment. Alignment components may comprise a Hirth joint formed by the teeth sets for self-alignment and positive engagement. The spring element may be overcome by force applied by the medical device handle. The system may accommodate usage misalignment that may occur when disconnecting, manually using, and reconnecting the handle. This motor adapter system may provide a more robust and precise connection mechanism between the medical device handle and the motor, addressing potential alignment issues and improving ease of use.

The present disclosure may comprise a system for connecting a catheter to motors that drive tip rotation and/or deflection knobs. Misalignment can occur when connecting the catheter to motors, especially after manual use, making reconnection difficult. Disclosed herein is a motor adapter system with alignment features to facilitate quick connection and disconnection in various conditions. Alignment features may include a tapered spline configuration. The tapered spline configuration may comprise a motor adapter with tapered splines on a motor shaft. The tapered spline configuration may comprise a deflection knob insert with features configured to mate with an associated tapered spline. The tapered spline configuration may allow power transmission while easing engagement.

Alignment features may comprise a spring loaded Hirth joint configuration. The spring loaded Hirth joint configuration may comprise a motor adapter on a motor shaft with teeth for a Hirth joint. The spring loaded Hirth joint configuration may comprise an intermediate adapter with matching teeth and splines. The spring loaded Hirth joint configuration may comprise a compression spring between the motor adapter and the intermediate adapter. The spring loaded Hirth joint configuration may comprise a deflection knob insert with mating splines. Force associated with the compression spring may be overcome by handle weight and/or magnets. The Hirth joints may be self-aligning for positive engagement.

The present disclosure may overcome misalignment from manual use and facilitate quick connection/disconnection. The present disclosure may improve robustness and precision of connection between the catheter and the motor(s) and enhance ease of use in clinical settings. The present disclosure may address alignment challenges in catheter-motor connections.

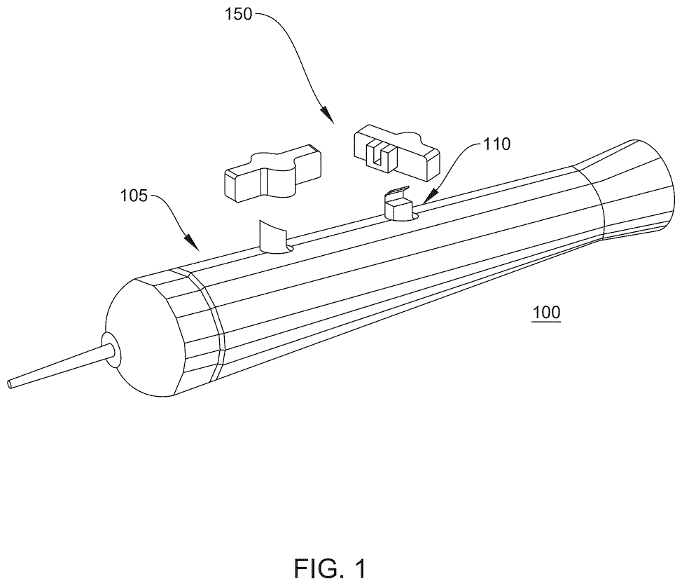

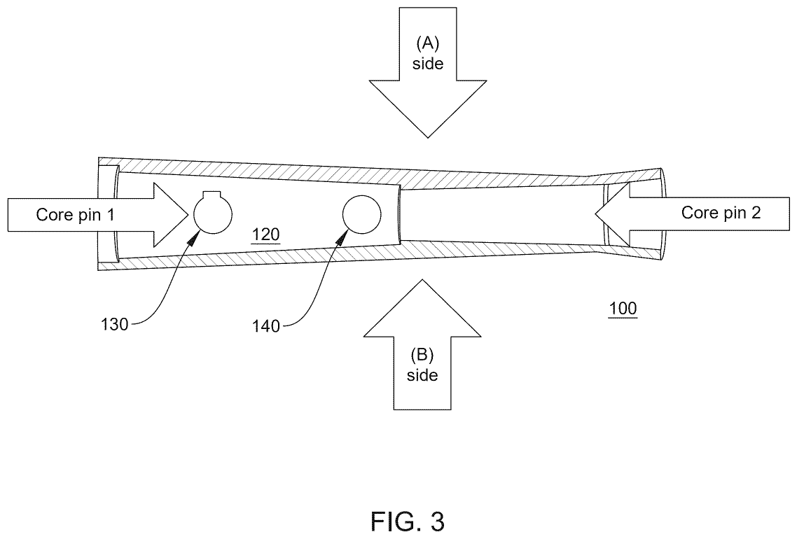

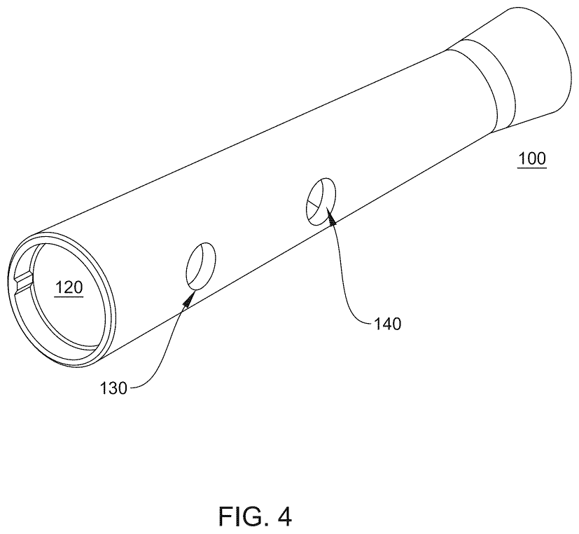

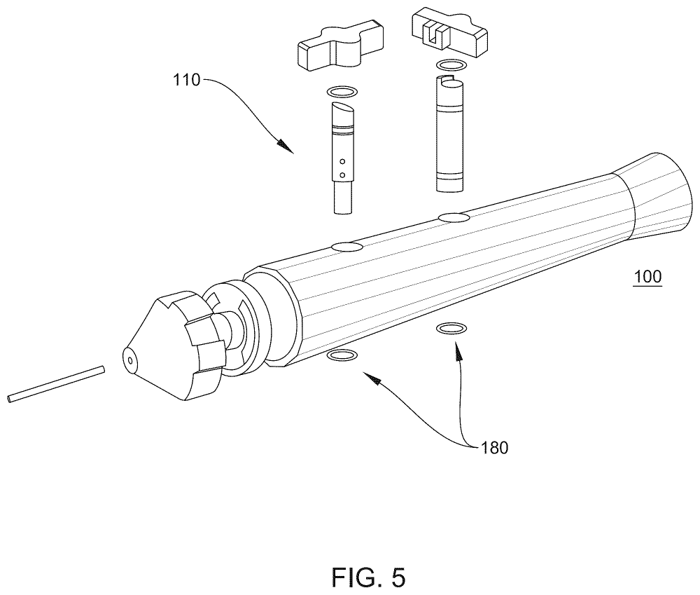

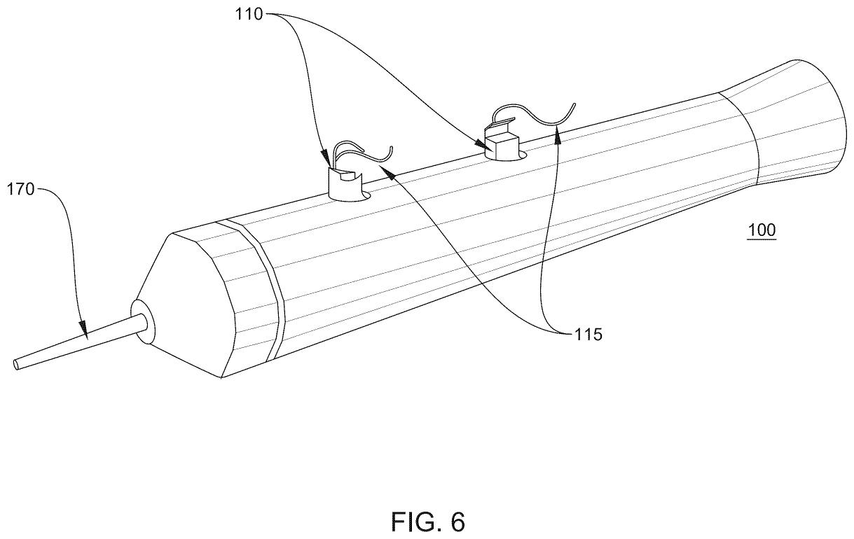

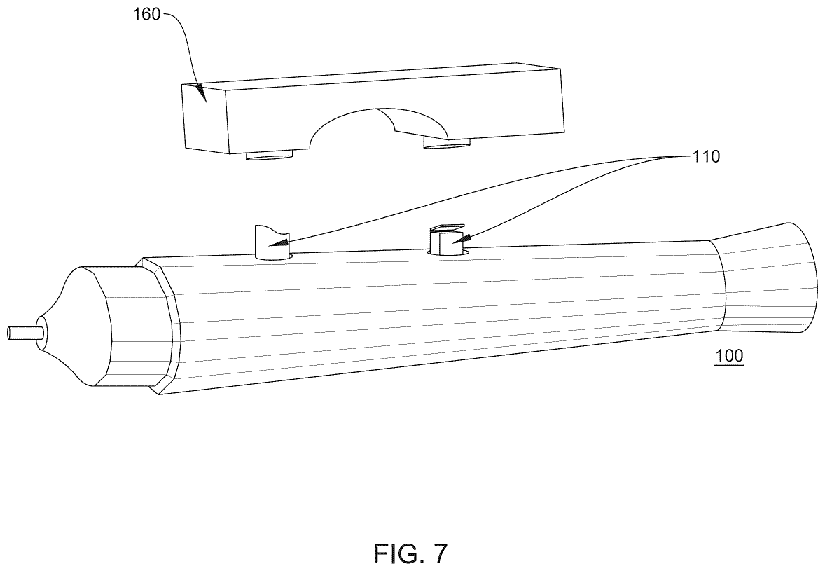

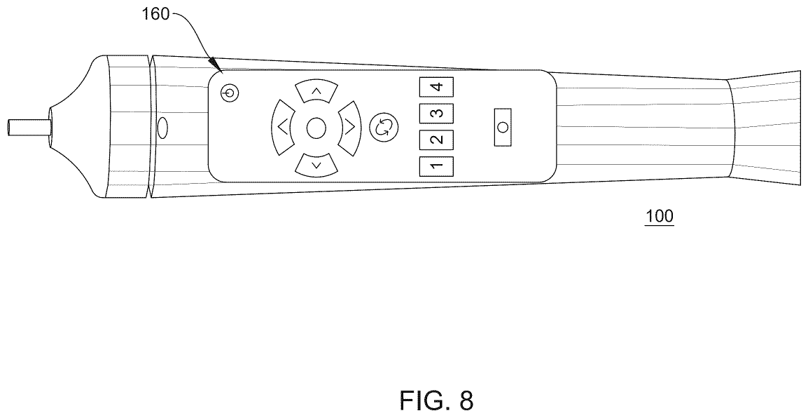

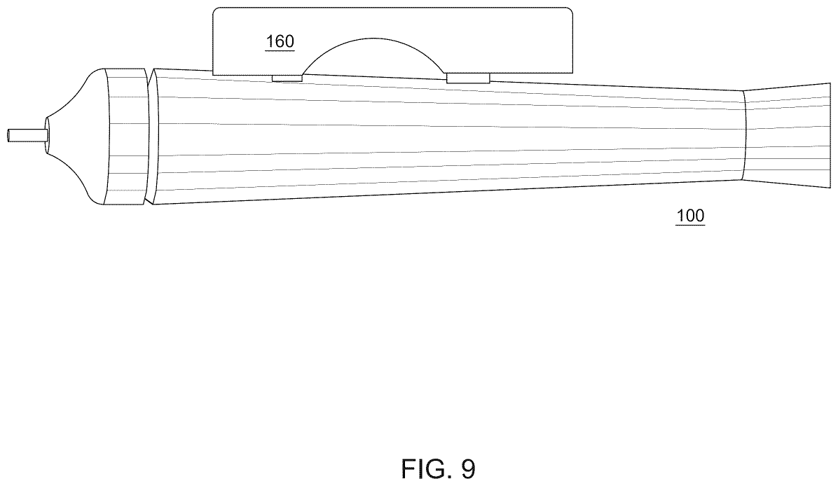

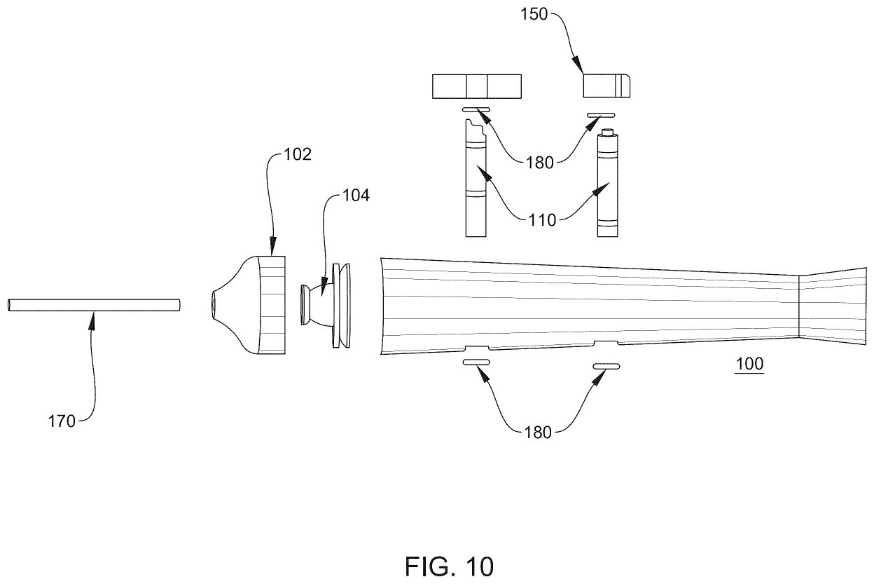

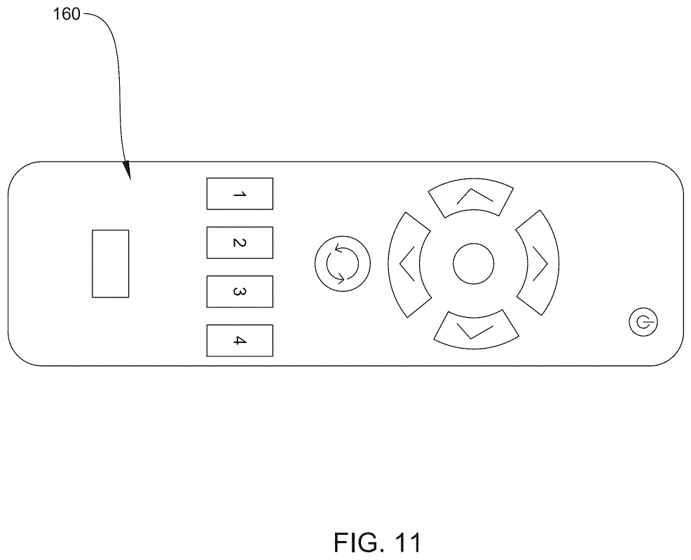

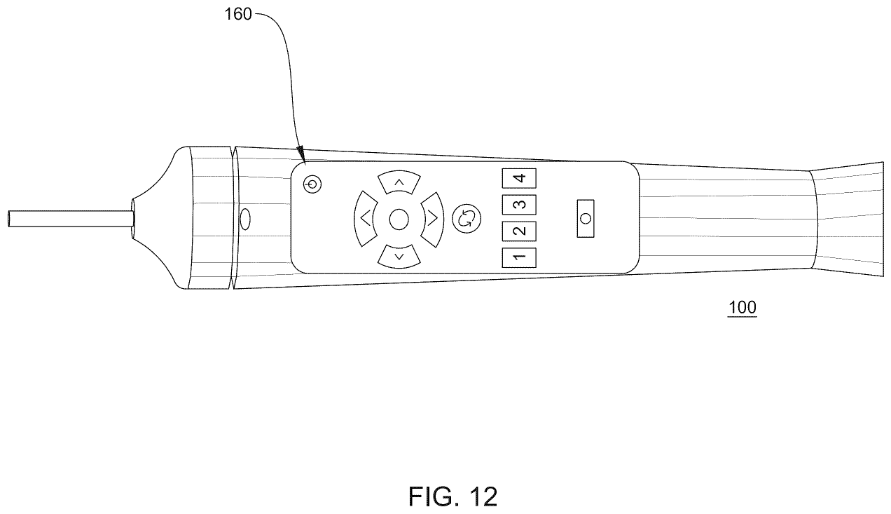

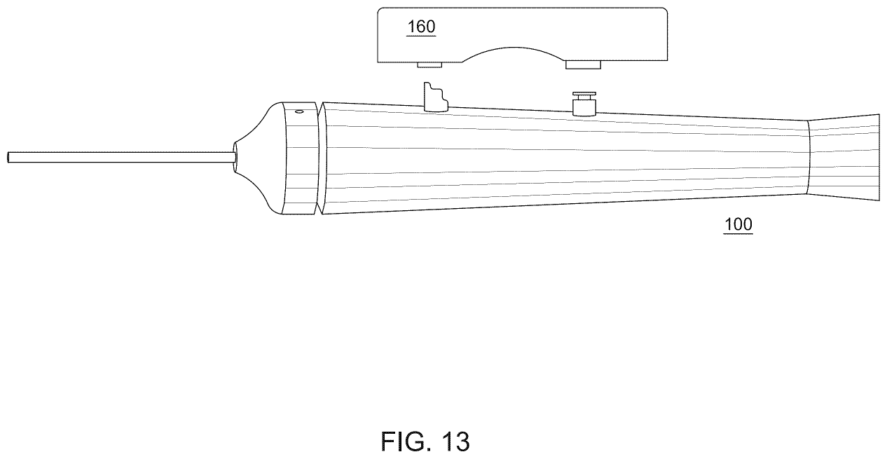

FIGS. 1-13 show an example catheter 100 described herein. The catheter 100 comprises a main body 105, one or more cartridges 110, and a shaft 170. The main body 105 may comprise a cavity 120 and a first aperture 130 and a second aperture 140 on a first side of the main body 105. The outer surface of the main body 105 may form a handle. The main body 105 may comprise a first partner aperture (not shown) and a second partner aperture (not shown) on a second side. A first cartridge of the one or more cartridges 110 may be configured to be disposed within the cavity 120, the first aperture 130, and the first partner aperture. A second cartridge of the one or more cartridges 110 may be configured to be disposed within the cavity 120, the second aperture 140, and the second partner aperture. The first side and the second side may be opposing sides of the main body 105. The main body 105 may comprise a single molded piece.

The main body 105 may be fully watertight. A watertight seal associated with the main body 105 may be capable of receiving an Ingress Protection X8 (IPX8) rating. The main body 105 may be submerged for reprocessing. The main body 105 may comprise a simplified assembly process. The main body 105 may comprise a modular design that may adapt to any catheter design. The main body 105 may facilitate 4-way steering capability with individual tip rotation. The main body 105 may facilitate 2-way steering capability with individual tip rotation. The main body 105 may comprise few component for assembly with the one or more cartridges 110.

The one or more cartridges 110 may be secured with one or more O-rings 180. An O-ring may be disposed at each aperture comprising one of the one or more cartridges, such as a first O-ring at the first aperture 130, a second O-ring at the first partner aperture, a third O-ring at the second aperture 140, and a fourth O-ring at the second partner aperture. The one or more O-rings may be disposed on an outer surface of the main body 105. The one or more O-rings 180 may create a watertight seal. A watertight seal associated with the one or more O-rings 180 and the main body 105 may be capable of receiving an Ingress Protection X8 (IPX8) rating. The one or more O-rings 180 may comprise a deflection auto-lock.

One or more pull lines 115 may be disposed in the one or more cartridges 110. The one or more cartridges 110 may be configured to rotate. Rotation of a cartridge may cause a change in tension applied to one or more pull lines. A change in tension applied to the one or more pull lines may cause a change in a state of the shaft 170. For example, a change in tension applied to the one or more pull lines may cause the shaft 170 to change deflection (e.g., deflect more, deflect less). As another example, a change in tension applied to the one or more pull lines may cause the shaft to rotate (e.g., rotate clockwise, rotate counter-clockwise). The one or more cartridges 110 may be designed to minimize components and reduce complexity for the one or more pull lines 115. The one or more pull lines 115 may be routed through the one or more cartridges 110 and locked into place.

The one or more cartridges 110 may be configured to receive one or more knobs 150. The one or more knobs 150 may be attached to the one or more cartridges 110 to facilitate manual manipulation of the one or more cartridges 110. The one or more cartridges 110 may comprise a locking mechanism. The one or more knobs 150 may be configured to snap into the locking mechanism of the one or more cartridges 110. The one or more knobs 150 may give a user an option to manually steer the shaft 170. The one or more cartridges 110 may facilitate side loading for deflection steering. The one or more cartridges 110 may facilitate steering similar to existing devices. The one or more cartridges 110 may comprise one or more keyed features to match keys for coupling with the one or more knobs 150.

The one or more cartridges 110 may be configured to receive an electronic controller 160. The electronic controller 160 may comprise one or more user interfaces. The one or more user interfaces may comprise one or more of a button, a knob, etc. The electronic controller 160 may be configured to snap into the locking mechanism of the one or more cartridges 110. The design may remove complexity and components from a single use catheter into the reusable electronic controller 160. The electronic controller 160 may be rechargeable. The electronic controller 160 may comprise one or more motors configured to interface with the one or more cartridges 110. The one or more cartridges 110 may comprise one or more keyed features to match keys for coupling with the electronic controller 160.

The catheter 100 may comprise and/or be in communication with an outer shaft base 102 and an inner shaft base 104. The outer shaft base 102 and/or the inner shaft base 104 may connect the shaft 170 with the main body 105.

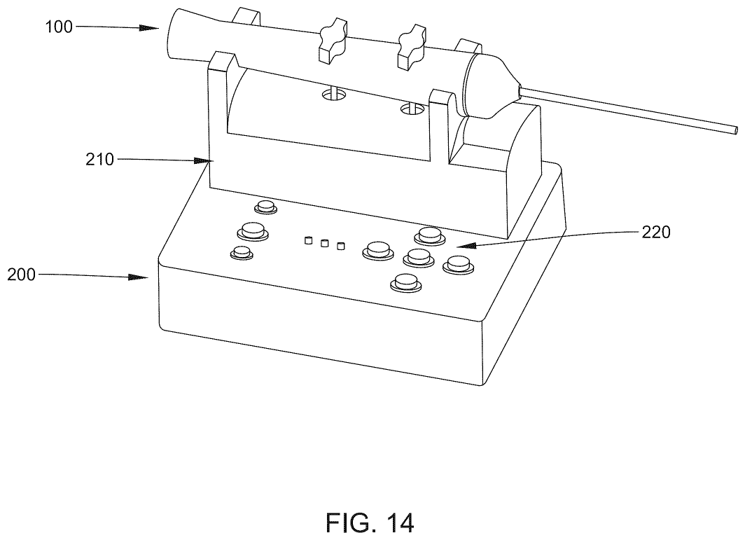

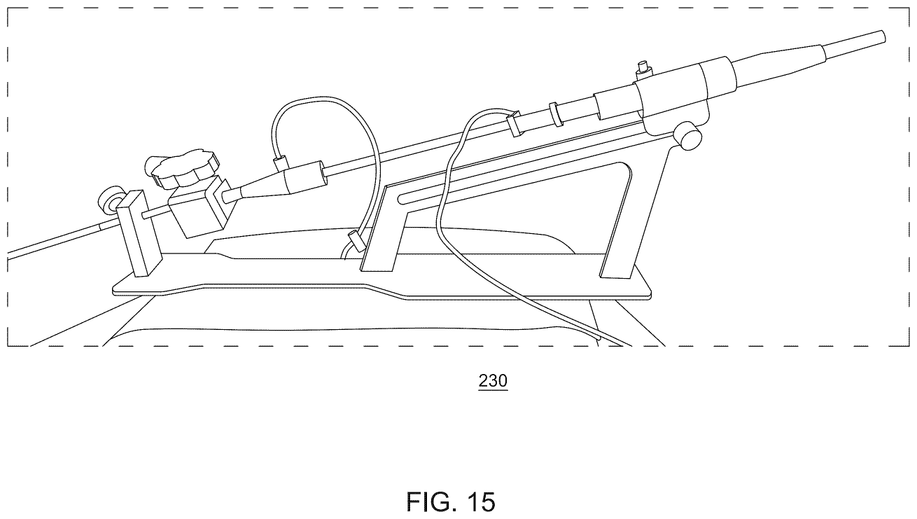

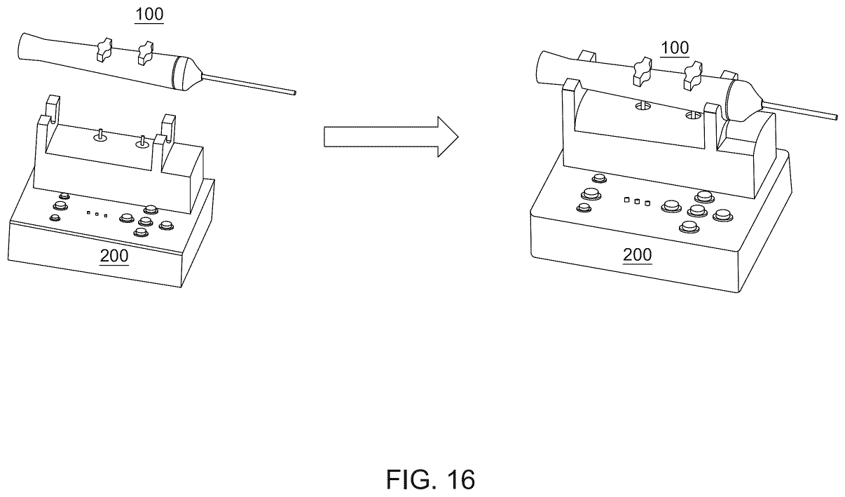

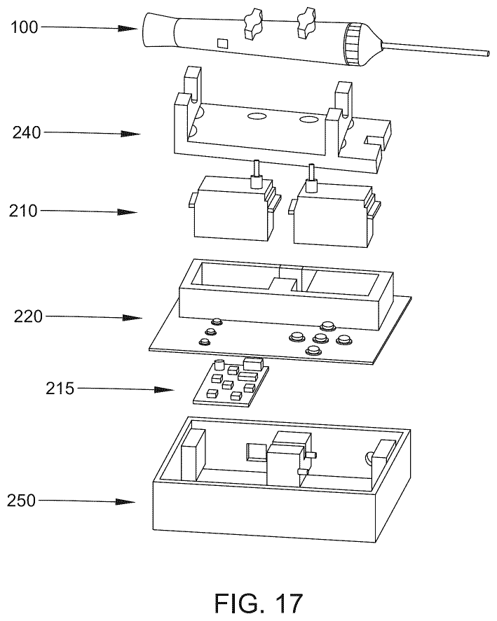

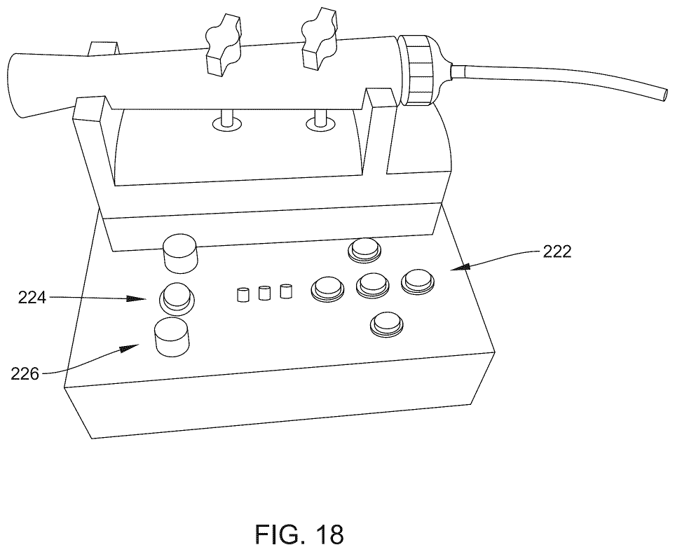

FIGS. 14-18 show an example stabilizer 200 described herein. The stabilizer 200 may comprise one or more servo motors 210, a controller interface 215, a user interface 220, a laminar stabilizer 230, a mount 240, and a base housing 250. Optionally, the stabilizer 200 may comprise a transceiver, such as a Bluetooth transceiver, a Wi-Fi transceiver, a cellular transceiver, etc. The stabilizer 200 may be configured to receive a catheter, such as the catheter 100. The stabilizer 200 may be rechargeable.

The one or more servo motors 210 may be configured to engage the one or more cartridges 110 of the catheter 100. The one or more servo motors 210 may be configured to cause the one or more cartridges 110 of the catheter 100 to rotate clockwise and/or counter-clockwise, and may be configured to rotate at various speeds and/or intervals.

The user interface 220 may comprise one or more control buttons 222, a home button 224, one or more knobs 226, and other user interface elements. Engagement with one of the one or more control buttons 222 may cause a change in position of the shaft 170 of the catheter 100. Engagement with the home button 224 may cause the shaft 170 of the catheter 100 to return to a predetermined home state. Engagement with the one or more knobs 226 may cause a change in position of the shaft 170 of the catheter 100. Engagement with the one or more control buttons 222, the home button 224, the one or more knobs 226, and/or the other user interface elements may cause the controller interface 215 to cause engagement of the one or more servo motors 210. The controller interface 215 may cause the one or more servo motors 210 to perform an action consistent with instructions indicated by the user interface elements engaged. The controller interface 215 may cause the one or more servo motors 210 to perform an action consistent with instructions received via the transceiver. The one or more knobs 226 may comprise a potentiometer control knob. The one or more control buttons 222 and/or the home button 224 may be engaged through pushing (pressing, etc.). The one or more knobs 226 may be engaged through rotation. The one or more control buttons 222 may be programmable.

The mount 240 may receive the catheter 100. The laminar stabilizer 230 may manipulate the mount 240 to stabilize the catheter 100. The base housing 250 may comprise some or all of the other components, such as the one or more servo motors 210, the controller interface 215, the user interface 220, the laminar stabilizer 230, and the mount 240.

A design of the catheter 100 may be simplified. As explained above, the catheter 100 may be controlled manually. As explained above, the catheter 100 may be controlled remotely. The catheter 100 may snap into the stabilizer 200. The catheter 100 may snap into the servo motors 210 of the stabilizer 200. The catheter 100 may snap into the mount 240 of the stabilizer 200. The stabilizer 200 may facilitate remote control of the catheter 100. The stabilizer 200 may comprise, via the laminar stabilizer 230, for example, tilt, translational displacement, feedback from imaging software, etc., to stabilize the catheter 100.

The stabilizer 200 may comprise translation control of the catheter 100. The stabilizer 200 may comprise tilt positions of the catheter 100. The stabilizer 200 may comprise auto focus of an image received from the image software. The stabilizer 200 may need little to no catheter manipulation from a physician.

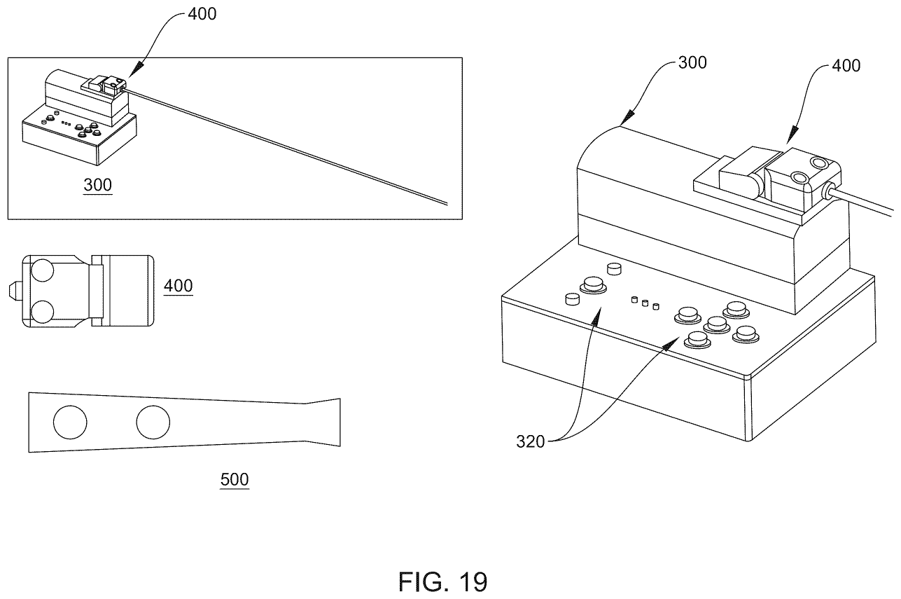

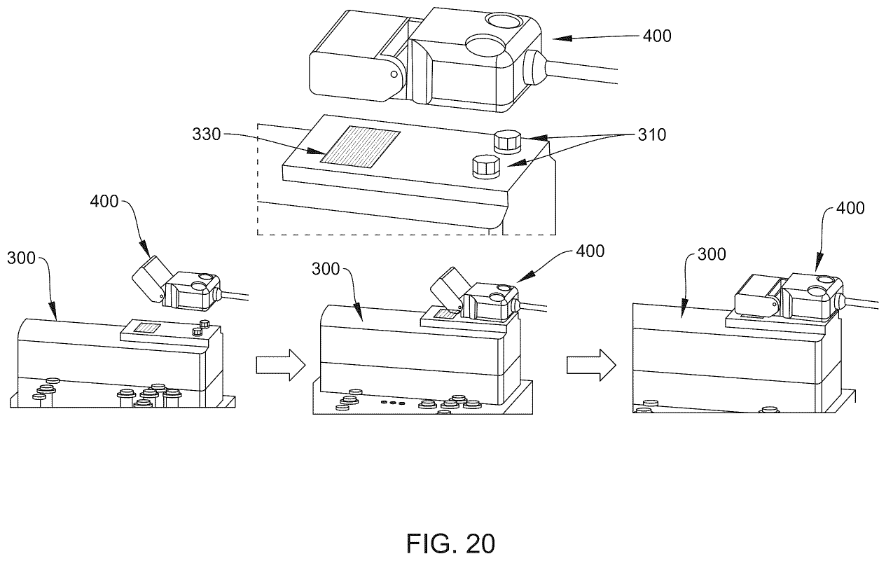

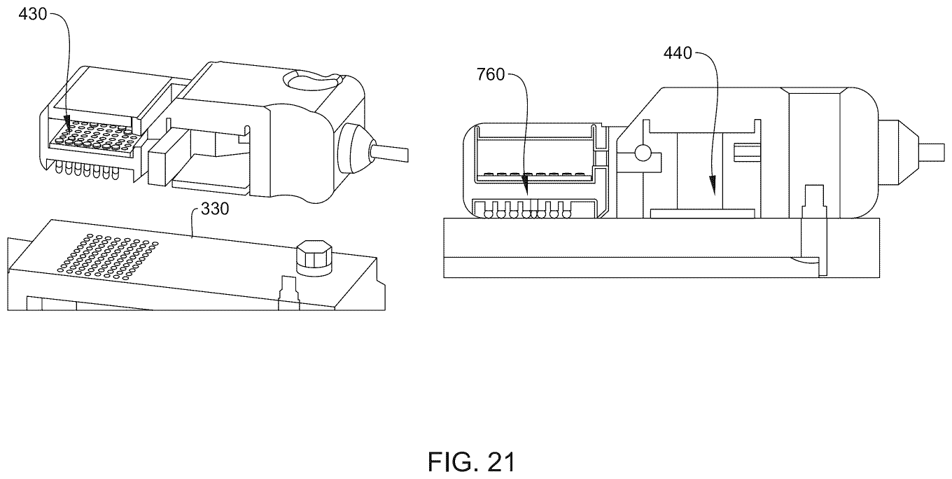

FIGS. 19-21 show an example catheter controller 300 described herein. The catheter controller 300 may be configured to couple with a catheter housing 400. The catheter controller 300 may be configured to stabilize the catheter housing 400. The catheter housing 400 may not have a handle, such as a handle 500 shown. The catheter controller 300 may comprise one or more drive motors 310, a user interface 320, and one or more connector pads 330.

One or more shafts associated with the one or more drive motors 310 may be received by one or more cavities of the catheter housing 400. Activation of the one or more drive motors 310 may cause the one or more shafts associated with the one or more drive motors 310 to rotate. Rotation of the one or more shafts associated with the one or more drive motors 310 may cause one or more drive motor shaft interfaces (not shown) associated with the catheter housing 400 to rotate. One or more pull lines (not shown) may be attached to the one or more drive motor shaft interfaces. Rotation of the one or more drive motor shaft interfaces may cause tension applied to one or more pull lines to change. Changing tension applied to one or more pull lines may cause a change in position of a shaft associated with the catheter controller 300. Causing a change in position of a shaft associated with the catheter controller 300 may comprise causing a change in deflection of the shaft and/or causing a change in rotation of the shaft.

The user interface 320 may comprise one or more control buttons, a home button, one or more knobs, and other user interface elements. Engagement with one of the one or more control buttons may cause a change in position of the shaft of the catheter housing 400. Engagement with the home button may cause the shaft of the catheter housing 400 to return to a predetermined home state. Engagement with the one or more knobs may cause a change in position of the shaft of the catheter housing 400. Engagement with the one or more control buttons, the home button, the one or more knobs, and/or the other user interface elements may cause engagement of the one or more servo motors 310. A controller interface may cause the one or more servo motors 310 to perform an action consistent with instructions indicated by the user interface elements engaged. The controller interface may cause the one or more servo motors 310 to perform an action consistent with instructions received via the transceiver. The one or more knobs may comprise a potentiometer control knob. The one or more control buttons and/or the home button may be engaged through pushing (pressing, etc.). The one or more knobs may be engaged through rotation. The one or more control buttons may be programmable.

The catheter controller 300 may comprise connector pads 330. The connector pads 330 may be over molded. The catheter housing 400 may comprise pogo pins 430. The catheter housing 400 may comprise electronic components 440. The electronic components 440 may comprise an electrically erasable programmable read-only memory (EEPROM), a printed circuit board (PCB) (including a secondary PCB for the EEPROM), a sensor, etc. The sensor may be one or more of a gyroscope, an accelerometer, a pressure sensor, etc.

A step of connecting the catheter housing 400 with the catheter controller 300 may comprise aligning the one or more drive motor shaft interfaces of the catheter housing 400 with the one or more shafts associated with the one or more drive motors 310 of the catheter controller 300. A step of connecting the catheter housing 400 with the catheter controller 300 may comprise inserting the one or more shafts associated with the one or more drive motors 310 of the catheter controller 300 into the one or more drive motor shaft interfaces of the catheter housing 400. A step of connecting the catheter housing 400 with the catheter controller 300 may comprise snapping pogo pins 430 of the catheter housing 400 into the connector pads 330 of the catheter controller 300. The combination of the pogo pins 430 of the catheter housing 400 and the connector pads 330 of the catheter controller 300 may result in a robust pogo pin connection 760.

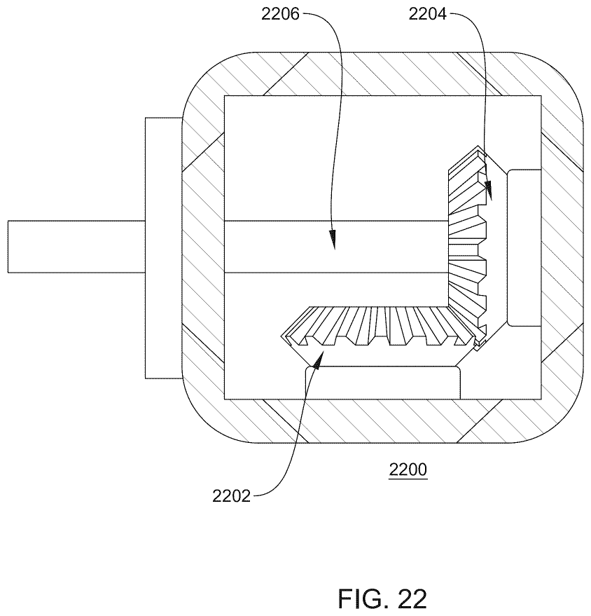

FIG. 22 illustrates a section view of the bevel gear mechanism 2200, which may be disposed in a handle shell of a medical device handle. The bevel gear mechanism 2200 may include a first bevel gear 2202 and a second bevel gear 2204 arranged in mechanical communication with each other. A shaft 2206 may be in communication with the second bevel gear 2204.

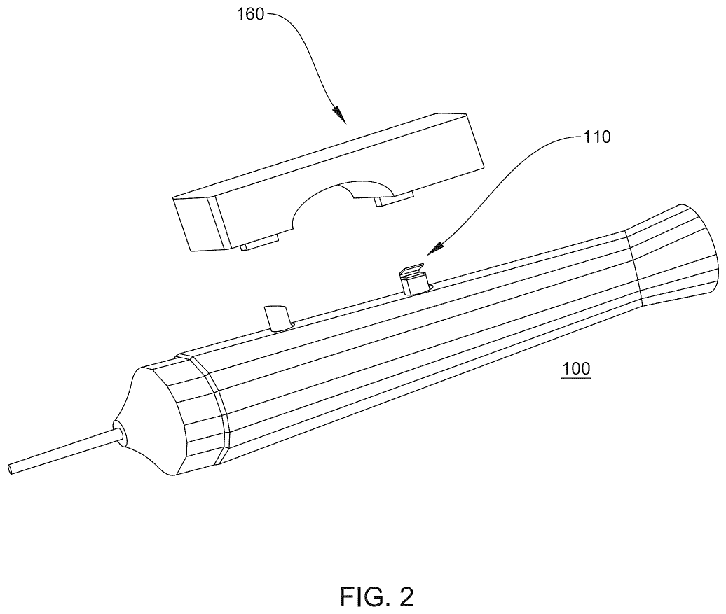

FIG. 2 shows a sectional view of the handle assembly incorporating the bevel gear mechanism 2200. The bevel gear mechanism 2200 may be configured to be inserted into a handle shell 2300. The bevel gear mechanism 2200 may be removably inserted into the handle shell 2300, allowing for potential maintenance or replacement.

The second bevel gear 2204 may be configured to connect to an outer shaft knob 2302. The second bevel gear 2204 may connect directly to the outer shaft knob 2302, or indirectly via at least one other component, such as the shaft 2206. The first bevel gear 2202 may include a mating interface configured to engage with a servo shaft 2301 when the handle assembly is positioned on a stabilizer. This configuration may allow the bevel gear mechanism 2200 to transmit rotational motion from the servo shaft 2301 to the outer shaft knob 2302.

The arrangement of the first bevel gear 2202 and second bevel gear 2204 within the bevel gear mechanism 2200 may enable tip rotation of a medical device. The mechanical communication between the first bevel gear 2202 and second bevel gear 2204 allows for the transfer of rotational motion between perpendicular axes, facilitating precise control of the medical device tip.

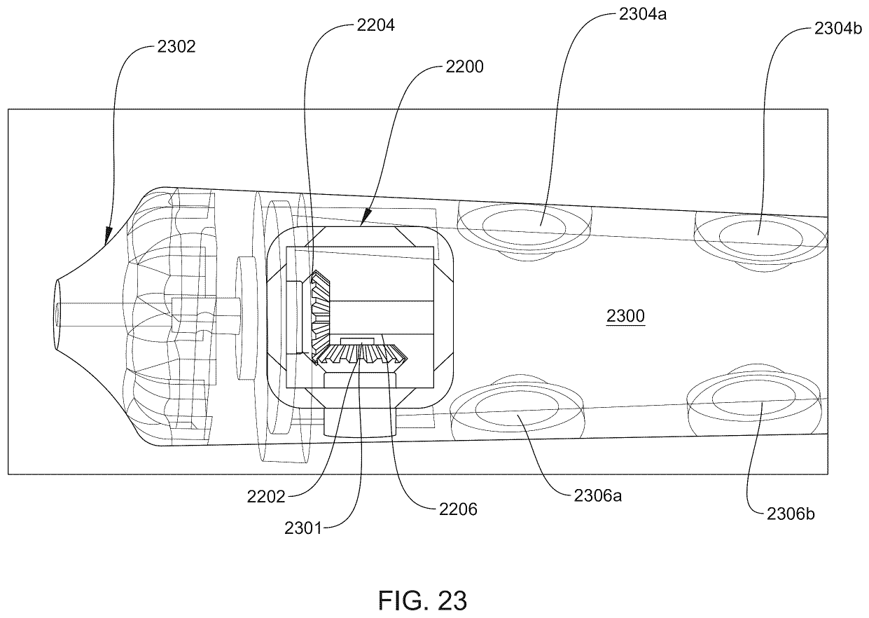

The handle shell 2300 may be designed as a one-piece structure that forms the main housing of the medical device handle assembly. FIG. 23 illustrates a sectional view of the handle shell 2300 and its internal components. The handle shell 2300 may include multiple cavities arranged along its length to accommodate various control components.

The handle shell 2300 may include a first deflection knob cavity 2304a and a corresponding first servo cavity 2306a and a second deflection knob cavity 2304b and a corresponding second servo cavity 2306b. The first deflection knob cavity 2304a and the corresponding first servo cavity 2306a may be disposed near opposite ends of the handle shell 2300 from the second deflection knob cavity 2304b and the corresponding second servo cavity 2306b, providing symmetrical placement for deflection control mechanisms. The servo cavities 2306a, 2306b may be designed to house servo components and may be arranged in a mirrored configuration within the handle shell 2300. The servo components may be disposed through the servo cavities 2306a, 2306b and the deflection knob cavities 2304a, 2304b and mate with deflection knobs of a handle. The servo components may be disposed through the servo cavities 2306a, 2306b and the deflection knobs may be disposed through the deflection knob cavities 2304a, 2304b and may mate with the servo components.

The arrangement of these cavities 2304a, 2304b, 2306a, 2306b within the handle shell 2300 may allow for efficient integration of control mechanisms. For example, the first deflection knob cavity 2304a and the second deflection knob cavity 2304b may be positioned to accommodate deflection control components, while the first servo cavity 2306a and the second servo cavity 2306b may be configured to house servo-driven mechanisms.

The handle shell 2300 may include water-tight seals. These seals may be incorporated into the design of the handle shell 2300 to enhance durability and maintain functionality of the internal components. The water-tight seals may help protect the bevel gear mechanism 2200, the first bevel gear 2202, the second bevel gear 2204, and other internal components from moisture or contaminants.

The handle shell 2300 may be configured to accommodate the shaft 2206 that extends from the bevel gear mechanism 2200. The outer shaft knob 2302 may be connected to one end of the handle shell 2300, allowing for manual control of the medical device.

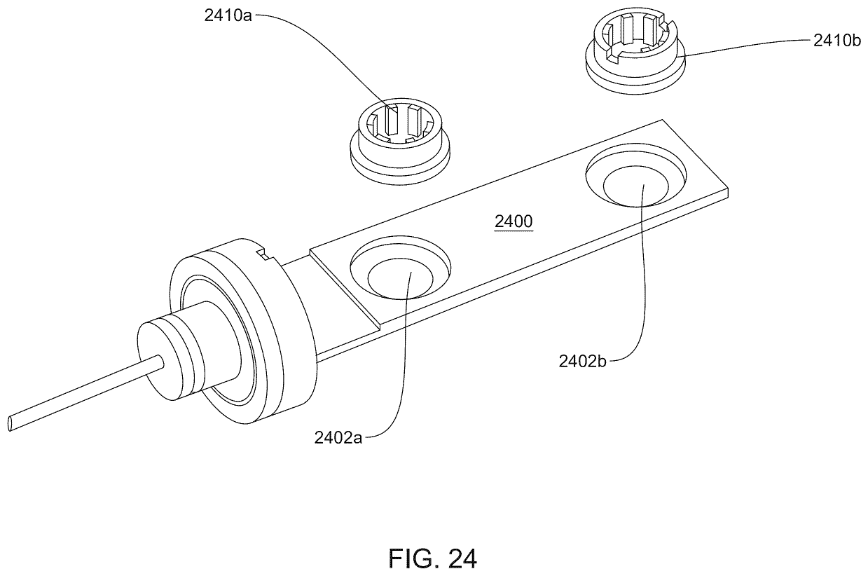

The medical device handle assembly may include a mounting plate 2400. FIG. 24 illustrates an exploded view of a mounting assembly, showing the mounting plate 2400 with a first spindle aperture 2402a and a second spindle aperture 2402b. These apertures 2402a, 2402b may be configured to receive a first spindle 2410a and a second spindle 2410b, respectively.

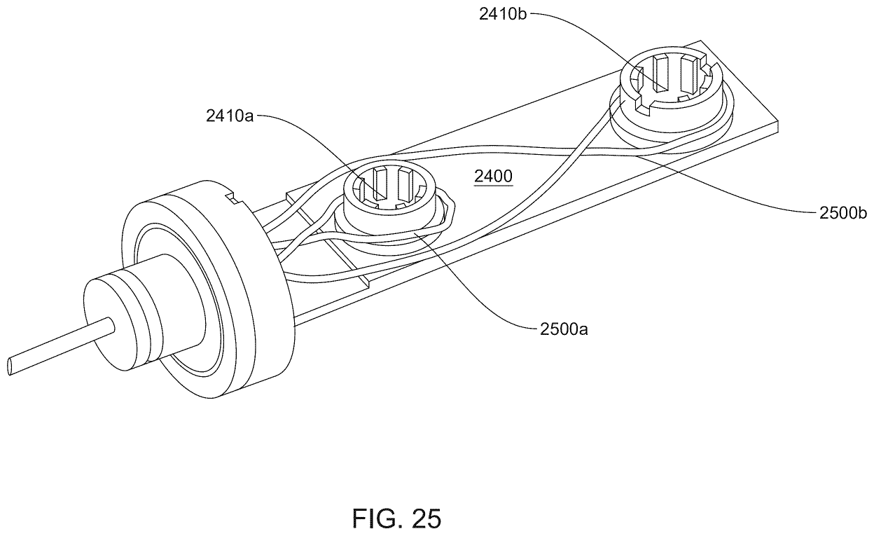

FIG. 25 provides a section view of the mounting assembly, demonstrating how the first spindle 2410a and the second spindle 2410b may be positioned along the mounting plate 2400. The first spindle 2410a may be configured to control deflection along a first axis, while the second spindle 2410b may be configured to control deflection along a second axis different from the first axis. The first spindle 2410a may be configured to control, for example, left and right deflection, and the second spindle 2410b may be configured to control, for example, up and down deflection. The first axis may be orthogonal the second axis. This arrangement may allow for orthogonal deflection control of a catheter or other medical device.

The mounting assembly may comprise a first pull line 2500a in communication with the first spindle 2410a and a second pull line 2500b in communication with the second spindle 2410b. Rotation of the first spindle 2410a in a first rotational direction may cause tension in the first pull line 2500a and cause an associated catheter to deflect in a first direction along the first axis. Rotation of the first spindle 2410a in a second rotational direction may cause tension in the first pull line 2500a and cause an associated catheter to deflect in a second direction along the first axis. Rotation of the second spindle 2410b in a first rotational direction may cause tension in the second pull line 2500b and cause an associated catheter to deflect in a first direction along the second axis. Rotation of the second spindle 2410b in a second rotational direction may cause tension in the second pull line 2500b and cause an associated catheter to deflect in a second direction along the second axis.

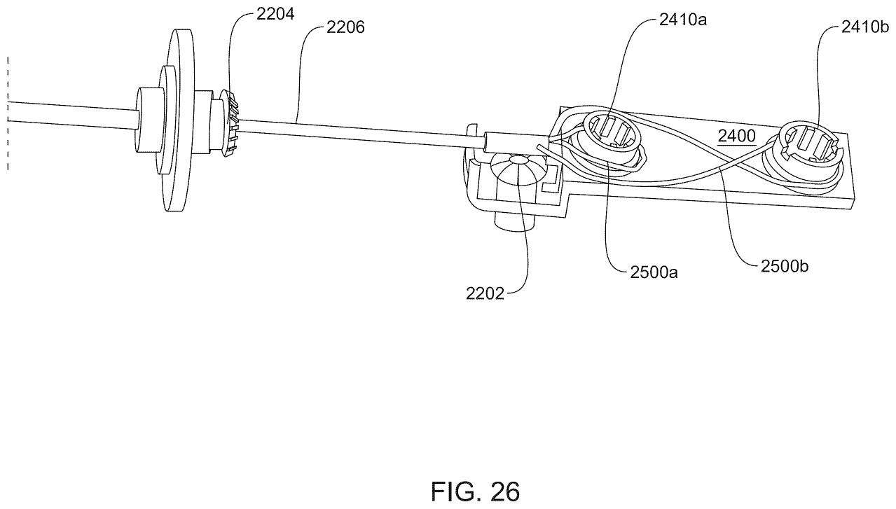

The mounting plate 2400 may serve as a mounting feature configured to accommodate the bevel gear mechanism 2200. FIG. 26 illustrates a side view of the mechanical assembly, showing how the mounting plate 2400 may interface with the first bevel gear 2202 and the second bevel gear 2204. This configuration may enable a tip rotation mechanism for the medical device. The tip rotation mechanism may be independent from movement along the first axis or the second axis facilitated by rotation of the first spindle 2410a or the second spindle 2410b, respectively.

The mounting plate 2400, along with the first spindle 2410a and the second spindle 2410b, may form part of a subassembly. This subassembly may be configured to be disposed (slide, etc.) in the handle shell 2300, as shown in FIG. 23. The subassembly may integrate pull lines and the tip rotation mechanism within a single cartridge structure.

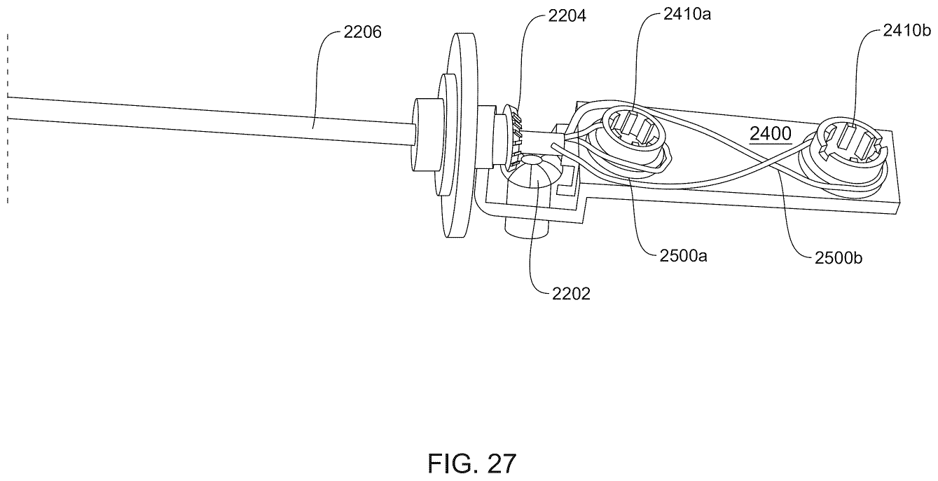

FIG. 27 depicts a side view of the mechanical assembly, showing the integration of the mounting plate 2400 with other components. A first pull line 2500a and a second pull line 2500b may be incorporated into the subassembly. These pull lines 2500a, 2500b may be configured for catheter deflection control, working in conjunction with the first spindle 2410a and the second spindle 2410b.

The mounting plate 2400 may be designed as a cartridge configured to receive the bevel gears 2202, 2204. This design may allow for efficient assembly and potential maintenance or replacement of components. The cartridge structure of the mounting plate 2400 may facilitate the integration of the pull lines 2500a, 2500b and the tip rotation mechanism into a single unit.

The arrangement of the first spindle 2410a and the second spindle 2410b on the mounting plate 2400 may provide a compact and efficient design for controlling multiple degrees of movement. The orthogonal positioning of these spindles 2410a, 2410b may allow for precise control over catheter deflection in different directions.

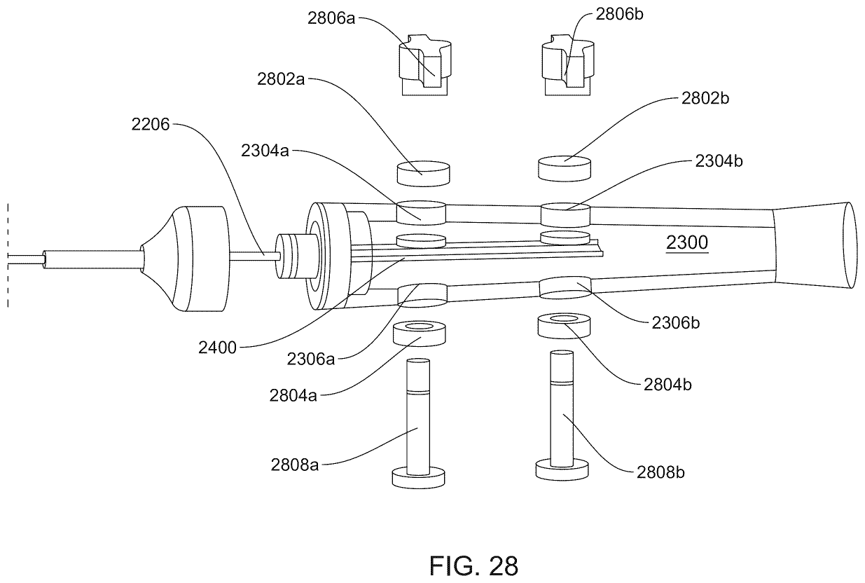

The medical device handle assembly may include various components that work together to provide control and functionality. FIG. 28 illustrates an exploded view of the handle assembly, showing the arrangement of these components within the handle shell 2300.

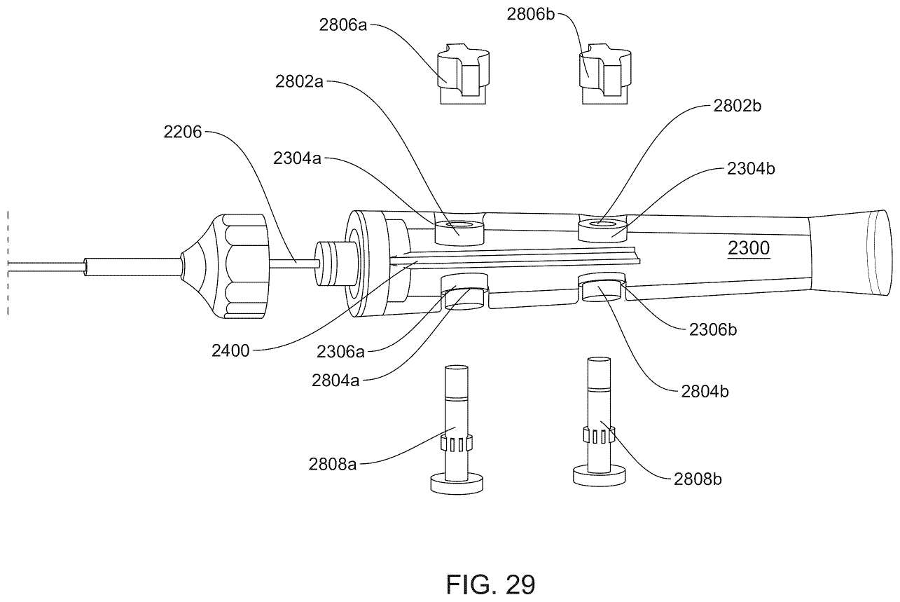

The handle assembly may include a first spindle cap nut 2802a and a second spindle cap nut 2802b. These spindle cap nuts 2802a, 2802b may be positioned at the top of the assembly, as shown in FIG. 29. The first spindle cap nut 2802a and the second spindle cap nut 2802b may be configured to secure other components within the handle shell 2300. The first spindle cap nut 2802a and the second spindle cap nut 2802b may act as water-tight seals.

The assembly may include a first spindle nut 2804a and a second spindle nut 2804b. These spindle nuts 2804a, 2804b may be positioned below the spindle cap nuts 2802a, 2802b, respectively. The first spindle nut 2804a and the second spindle nut 2804b may serve to adjust or maintain tension on other components within the assembly. The first spindle nut 2804a and the second spindle nut 2804b may act as water-tight seals.

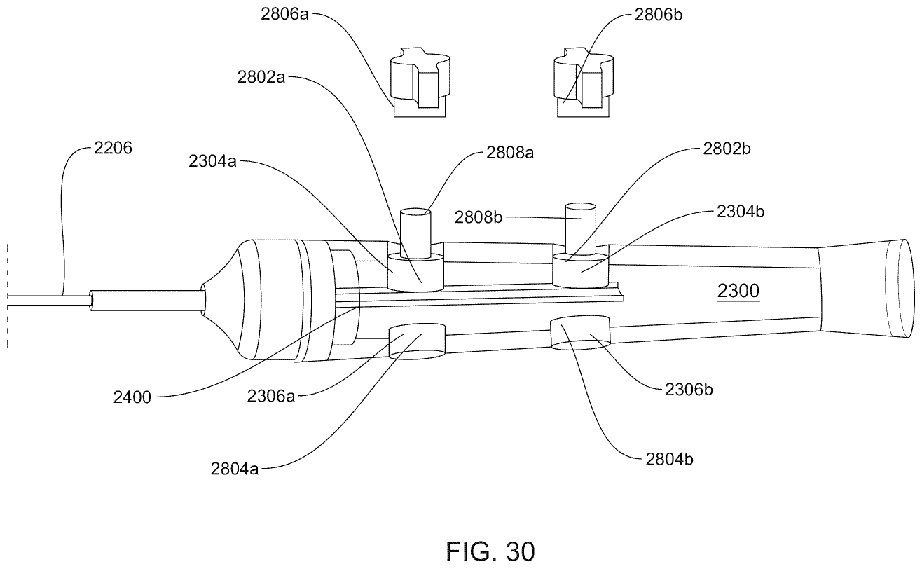

FIG. 30 depicts a section view of the handle assembly, revealing the internal arrangement of components. The handle assembly may include a first spindle 2808a and a second spindle 2808b. FIG. 30 shows how these spindles 2808a, 2808b may be positioned within the handle shell 2300. The first spindle 2808a and the second spindle 2808b may be configured to interact with the first pull line 2500a and the second pull line 2500b, respectively, to control deflection of a medical device.

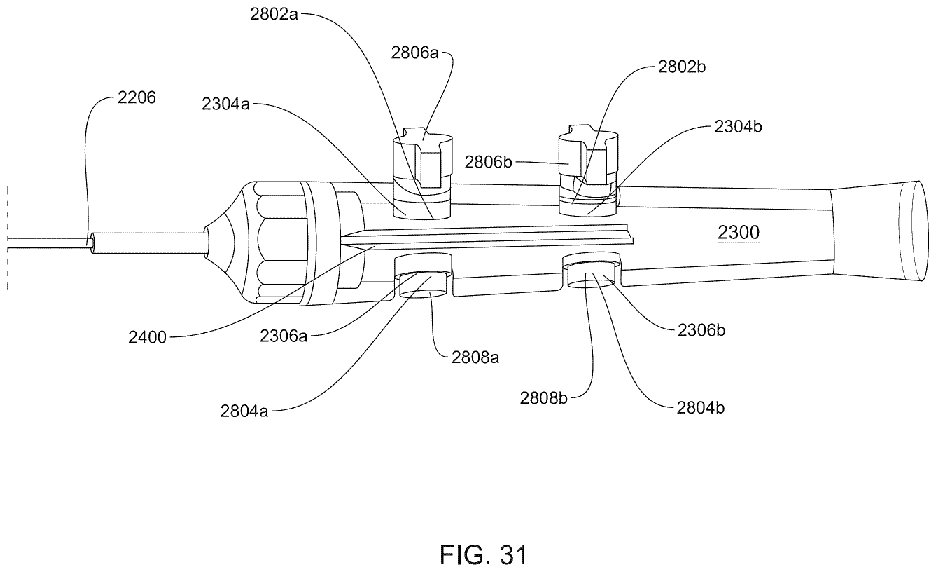

The handle assembly may also incorporate a first spindle cap 2806a and a second spindle cap 2806b. As illustrated in FIG. 31, these spindle caps 2806a, 2806b may be positioned on top of the spindles 2808a, 2808b and adjacent the spindle cap nuts 2802a, 2802b. The first spindle cap 2806a and the second spindle cap 2806b may provide an interface between the spindle components and other elements of the handle assembly.

The arrangement of these components within and/or on the handle shell 2300 may allow for a compact and efficient design. The components may be pre-assembled as a single unit prior to disposition into the handle shell 2300. This pre-assembly may facilitate easier manufacturing and potential maintenance or replacement of the internal components.

The handle assembly may include water-tight seals. These seals may be incorporated at various points within the assembly to provide durability and maintain functionality of the internal components. For example, water-tight seals may be placed around the first spindle cap 2806a and the second spindle cap 2806b where they interface with the handle shell 2300.

The configuration of these components within the handle shell 2300 may allow for efficient integration with other elements of the medical device handle assembly. For instance, the first spindle 2808a and the second spindle 2808b may be positioned to align with the first deflection knob cavity 2304a and the second deflection knob cavity 2304b, respectively. Similarly, the arrangement may accommodate the bevel gear mechanism 2200, including the first bevel gear 2202 and the second bevel gear 2204, within the handle shell 2300.

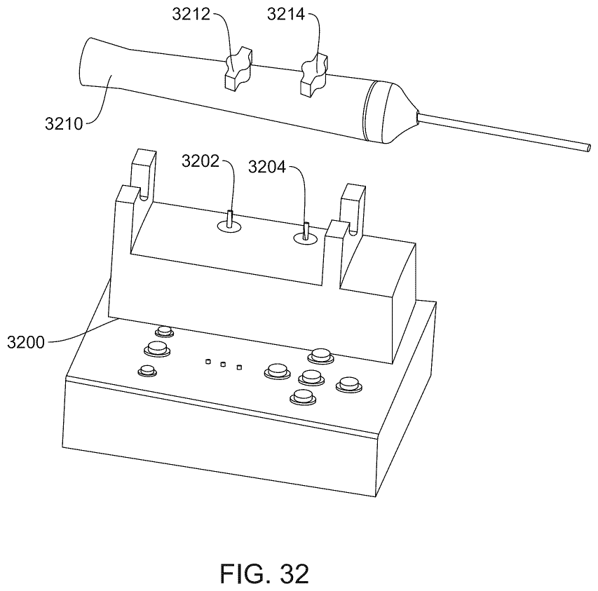

The medical device handle assembly may include a mount (stabilizer, dock, station, etc.) 3200 configured to interface with a catheter 3210. FIG. 32 illustrates an view of a mounting assembly that includes the mount 3200 and associated components.

The mount 3200 may serve as a base structure for connecting the catheter 3210 to other components of the medical device system. The mount 3200 may include a first mount spindle 3202 and a second mount spindle 3204 extending from a surface of the mount 3200. The first mount spindle 3202 and the second mount spindle 3204 may be configured to engage with corresponding components of the catheter 3210.

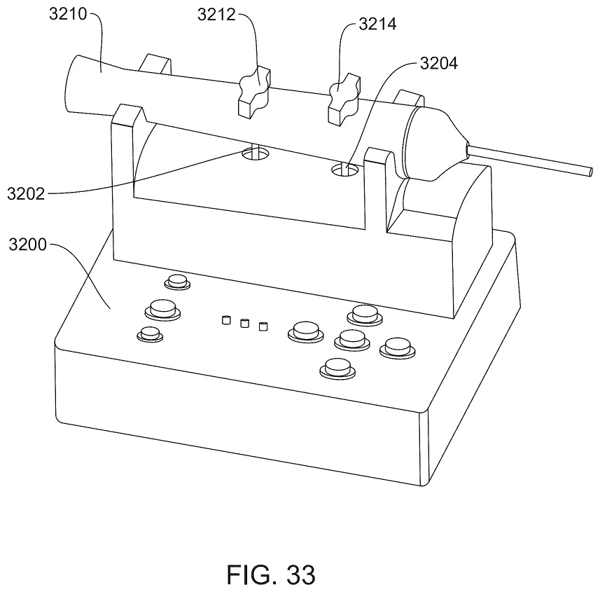

FIG. 33 shows a perspective view of the mount 3200, demonstrating how the catheter 3210 may be positioned to interface with the mount 3200. The catheter 3210 may include a first spindle cap 3212 and a second spindle cap 3214. The first spindle cap 3212 may be configured to engage with the first mount spindle 3202, while the second spindle cap 3214 may be configured to engage with the second mount spindle 3204.

The arrangement of the first mount spindle 3202 and the second mount spindle 3204 on the mount 3200 may allow for secure attachment of the catheter 3210. This configuration may provide stability and proper alignment between the catheter 3210 and other components of the medical device system.

The first spindle cap 3212 and the second spindle cap 3214 may be designed to facilitate quick connection and disconnection of the catheter 3210 from the mount 3200. The spindle caps 3212, 3214 may include features that allow for easy alignment with the corresponding mount spindles 3202, 3204.

The mount 3200 may be designed to work in conjunction with other components of the medical device handle assembly. For example, the mount 3200 may be configured to interface with the handle shell 2300, allowing for integration of the catheter 3210 with the bevel gear mechanism 2200 and other control components.

The first mount spindle 3202 and the second mount spindle 3204 may be positioned to align with the first spindle 2808a and the second spindle 2808b within the handle assembly. This alignment may allow for the transfer of mechanical motion from the mount 3200 to the catheter 3210, enabling precise control of catheter movements.

The mount 3200 may provide a means for connecting the catheter 3210 to servo-driven mechanisms or other control systems. The mount 3200 may include features that allow for the transmission of rotational or linear motion from motors or other actuators to the catheter 3210 through the first mount spindle 3202 and the second mount spindle 3204.

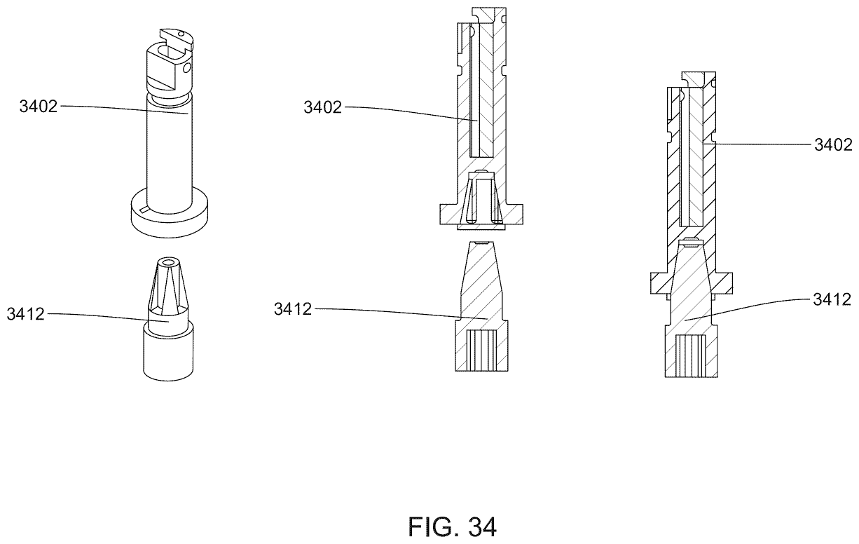

The medical device handle assembly may include a motor shaft connection assembly. FIG. 34 illustrates a view of the motor shaft connection assembly shown from multiple angles. The motor shaft connection assembly may include a deflection knob insert 3402 and a motor shaft connector 3412. The motor shaft connector 3412 may be configured as a tapered spline, and the deflection knob insert 3402 may be configured to mate with the tapered spline.

The deflection knob insert 3402 may be configured to mate with the motor shaft connector 3412. The deflection knob insert 3402 may appear as a cylindrical component with specific mating features at one end. The motor shaft connector 3412 may be shown as an elongated component with corresponding mating features designed to engage with the deflection knob insert 3402. The catheter 3210 may comprise one or more deflection knob insert(s) 3402.

The motor shaft connector 3412 may be configured to be positioned on a motor shaft. The motor shaft may be part of a servo-driven mechanism. The servo-driven mechanism may be configured to provide precise control of tip rotation for the medical device. The first mount spindle 3202 and/or the second mount spindle 3204 may comprise a motor shaft connector 3412.

A mating interface between the deflection knob insert 3402 and the motor shaft connector 3412 may include a safety feature. This safety feature may be configured to allow slipping when a predetermined force threshold is exceeded. This configuration may help prevent damage to the medical device or injury to a patient in case of excessive force application.

The deflection knob insert 3402 may be designed to interface with other components of the medical device handle assembly. For example, the deflection knob insert 3402 may be connected to the bevel gear mechanism 2200, allowing for the transfer of rotational motion from the servo-driven mechanism to the outer shaft knob 2302.

The motor shaft connector 3412 may be designed to accommodate potential misalignment between the deflection knob insert 3402 and the motor shaft. This design may facilitate easier connection and disconnection of the medical device handle assembly from the servo-driven mechanism.

The arrangement of the deflection knob insert 3402 and the motor shaft connector 3412 may allow for efficient power transmission while providing safety features and ease of use. This configuration may enable precise control of the medical device tip rotation while maintaining flexibility in the connection between the handle assembly and the servo-driven mechanism.

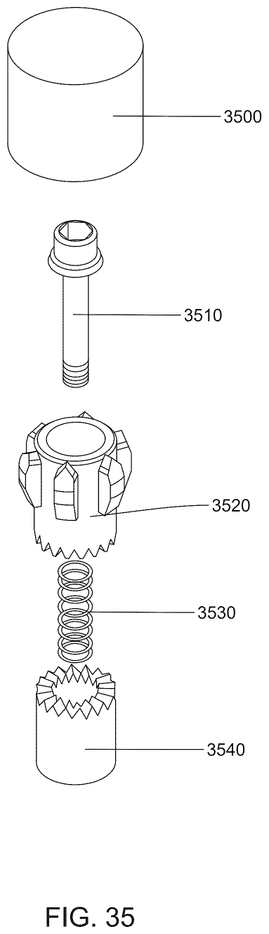

The medical device handle assembly may include a motor adapter system. FIG. 35 illustrates an exploded view of a motor adapter assembly. The motor adapter assembly may include a deflection knob insert 3500, a fastener 3510, an intermediate adapter 3520, a compression spring 3530, and a motor adapter 3540. The motor adapter assembly may comprise a spring loaded Hirth joint arrangement.

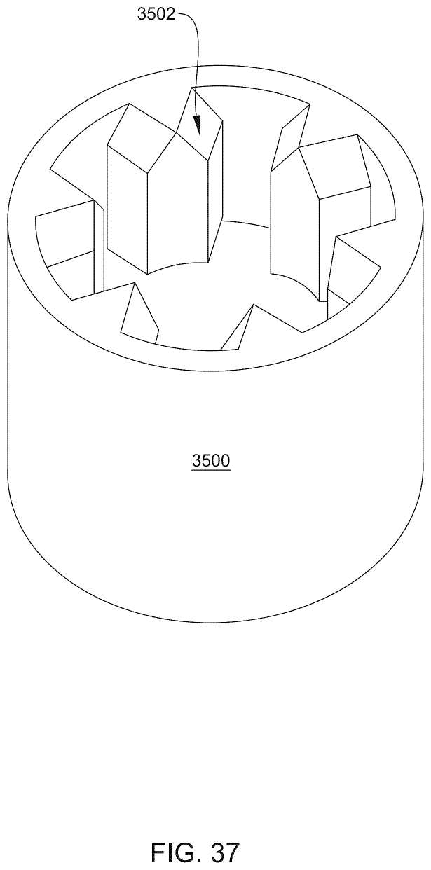

The deflection knob insert 3500 may be configured to mate with the motor adapter 3540. FIG. 37 shows a section view of the deflection knob insert 3500, which may include a chamfered configuration 3502 at its interior surface. The chamfered configuration 3502 may facilitate easier alignment and connection with other components of the motor adapter assembly. The catheter 3210 may comprise one or more deflection knob insert(s) 3500.

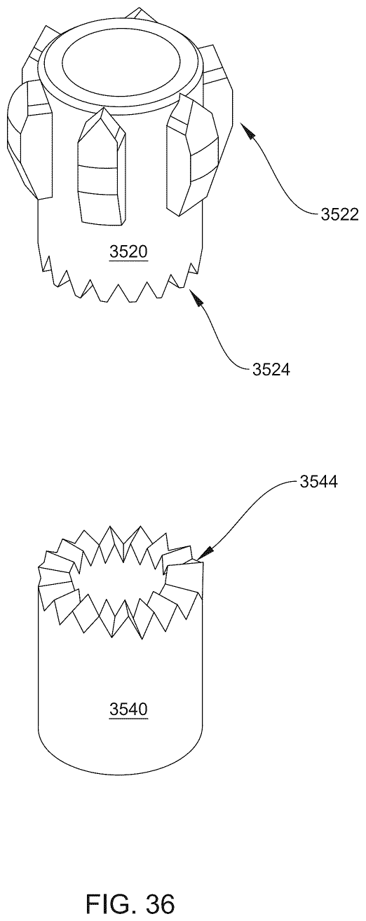

The motor adapter 3540 may be configured to be positioned on a motor shaft. FIG. 36 illustrates a section view of the motor adapter 3540 and the intermediate adapter 3520. The motor adapter 3540 may include a second serrated rim 3544 configured to mate with a first serrated rim 3524 of the intermediate adapter 3520. The first serrated rim 3524 and the second serrated rim 3544 may form a Hirth joint for self-alignment and positive engagement. The first mount spindle 3202 and/or the second mount spindle 3204 may comprise a motor adapter 3540.

The intermediate adapter 3520 may include splines with chamfers 3522 along its outer surface. These splines with chamfers 3522 may be designed to allow power transmission while facilitating easier sliding engagement with the deflection knob insert 3500. The chamfered features of the splines may aid in aligning the components during assembly.

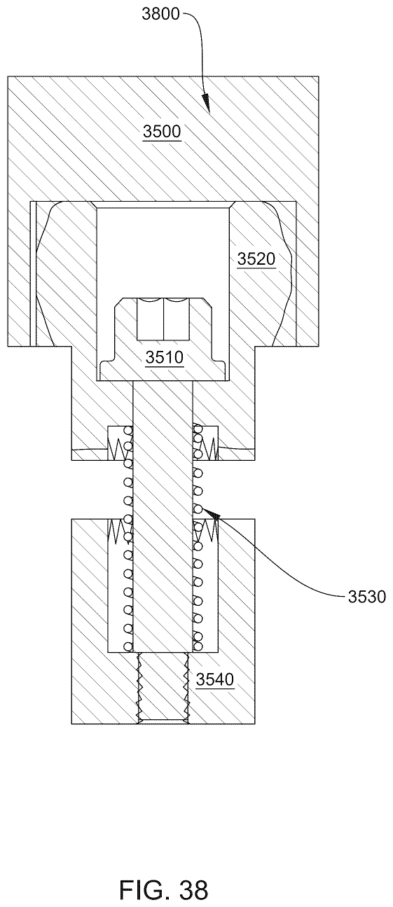

FIG. 38 shows a section view of the motor adapter assembly under an applied downward force 3800. The compression spring 3530 may be situated between the intermediate adapter 3520 and the motor adapter 3540. The compression spring 3530 may be configured to bias the intermediate adapter 3520 away from the motor adapter 3540. The downward force 3800 may overcome the spring force, allowing the intermediate adapter 3520 to engage with the motor adapter 3540.

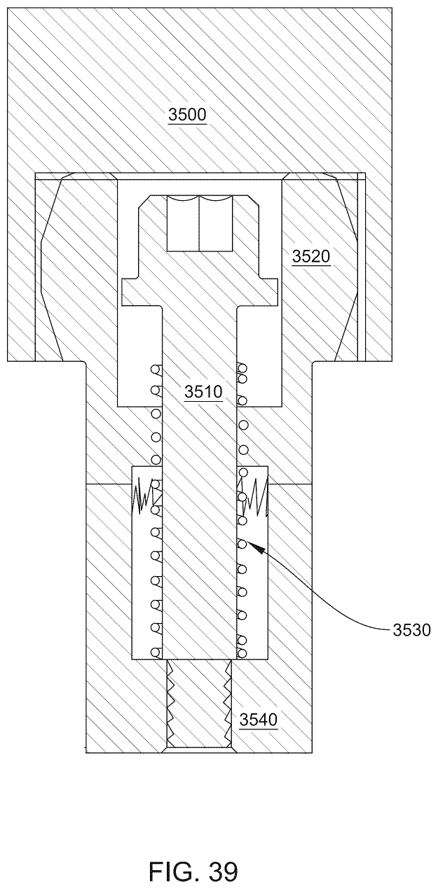

The fastener 3510 may extend through the central axis of the assembly, as shown in FIG. 39. The intermediate adapter 3520 may rotate freely on the fastener 3510, allowing for easier alignment with the deflection knob insert 3500.

The motor adapter system may include at least one alignment component to facilitate connection between the deflection knob insert 3500 and the motor adapter 3540 and to prevent a connection when a misalignment is present. These alignment features may allow for quick connection and disconnection of the medical device handle in various operational conditions. For example, the motor adapter 3540 may accommodate and fix a usage misalignment that may occur when a user disconnects the medical device handle, uses the medical device handle manually, and then attempts to reconnect the medical device handle to the motor.

Magnets may be used to help overcome the spring force in the motor adapter system. The magnetic force may work in conjunction with the downward force 3800 to engage the intermediate adapter 3520 with the motor adapter 3540.

The arrangement of these components within the motor adapter assembly may provide a robust system for connecting the medical device handle to a motor while accommodating and fixing potential misalignments. The combination of the chamfered configuration 3502, splines with chamfers 3522, serrated rims 3524, 3544, and compression spring 3530 may work together to ensure proper alignment and engagement between the deflection knob insert 3500 and the motor adapter 3540.

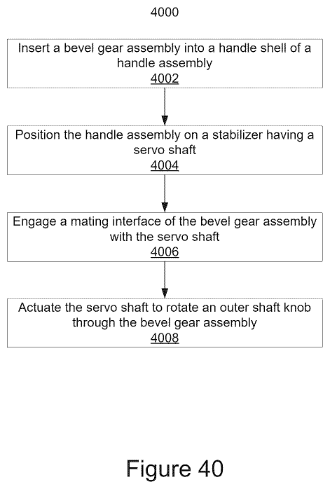

FIG. 40 illustrates methods 4000 for assembling and operating a medical device handle assembly. The methods 4000 may include a sequence of operations regarding the medical device handle assembly.

A bevel gear assembly may be inserted into a handle shell (block 4002). The bevel gear mechanism 2200 may include the first bevel gear 2202 and the second bevel gear 2204, which may be configured to enable tip rotation of a medical device. The first bevel gear may be in communication with an outer shaft knob.

The handle assembly may be disposed on a stabilizer having a servo shaft (block 4004). At least one deflection knob insert of the handle assembly may be in communication with at least one motor shaft connector and/or motor adapter of the stabilizer. This configuration may allow for precise control of the medical device tip rotation through servo-driven mechanisms.

A mating interface of the bevel gear assembly may be engaged with the servo shaft (block 4006). The second bevel gear may be engaged with the servo shaft.

The servo shaft may be actuated to rotate the outer shaft knob through the bevel gear assembly (block 4008). Rotation of the servo shaft may cause rotation of the second bevel gear, which in turn may cause rotation of the first bevel gear, which in turn may cause rotation of the outer shaft knob. This step may enable the transmission of rotational motion from the servo shaft to the outer shaft knob 2302, facilitating precise control of the medical device.

Although FIG. 40 shows example blocks of methods 4000, in some implementations, methods 4000 may include additional blocks, fewer blocks, different blocks, or differently arranged blocks than those depicted in FIG. 40. Additionally, or alternatively, two or more of the blocks of methods 4000 may be performed in parallel.

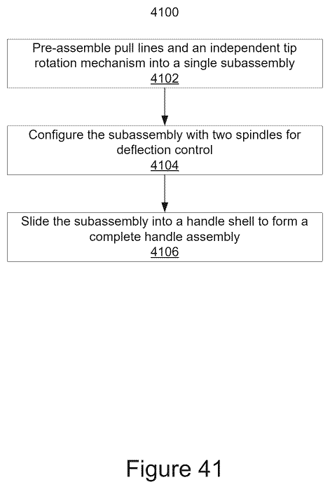

FIG. 41 illustrates methods 4100 for assembling and operating a medical device handle assembly. The methods 4100 may include a sequence of operations regarding the medical device handle assembly.

Pull lines and an independent tip rotation mechanism may be pre-assembled into a single subassembly (block 4102). This subassembly may integrate the first pull line 2500a, the second pull line 2500b, and components of the bevel gear mechanism 2200.

The subassembly may be configured with two spindles for deflection control (block 4104). Each pull line may be coupled to one of the spindles. One of the spindles may control deflection along a first axis and the other spindle may control deflection along a second axis different from the first axis. The first axis may be orthogonal the second axis.

The subassembly may be slid into a handle shell to form a complete handle assembly (block 4106). This step may allow for efficient integration of the pre-assembled components into the handle shell 2300.

Although FIG. 41 shows example blocks of methods 4100, in some implementations, methods 4100 may include additional blocks, fewer blocks, different blocks, or differently arranged blocks than those depicted in FIG. 41. Additionally, or alternatively, two or more of the blocks of methods 4100 may be performed in parallel.

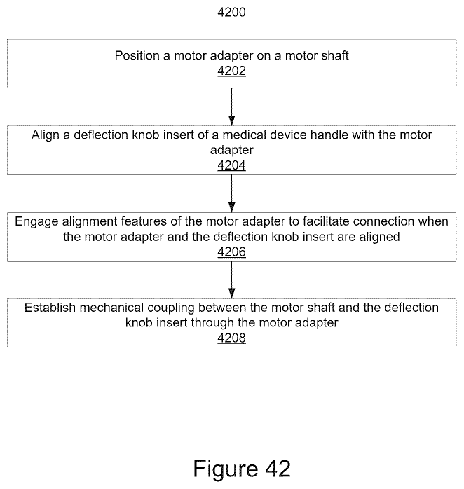

FIG. 42 illustrates methods 4200 for assembling and operating a medical device handle assembly. The methods 4200 may include a sequence of operations for establishing a secure connection between a motor adapter and the medical device handle assembly.

A motor adapter may be positioned on a motor shaft (block 4202). The motor adapter may be configured to interface with other components of the medical device handle assembly.

A deflection knob insert of a medical device handle may be aligned with the motor adapter (block 4204). The deflection knob insert may include a chamfered configuration to facilitate easier alignment and connection.

Alignment features of the motor adapter may be engaged to facilitate connection when the motor adapter and the deflection knob insert are aligned (block 4206). The engaging the alignment features may comprise disposing the deflection knob insert onto tapered splines of the motor adapter. The engaging the alignment features may comprise overcoming a spring force to engage complementary teeth of an intermediate adapter with teeth of the motor adapter and aligning splined interfaces through chamfered features.

Mechanical coupling may be established between the motor shaft and the deflection knob insert through the motor adapter (block 4208). This step may enable efficient power transmission while providing safety features and ease of use.

Although FIG. 42 shows example blocks of methods 4200, in some implementations, methods 4200 may include additional blocks, fewer blocks, different blocks, or differently arranged blocks than those depicted in FIG. 42. Additionally, or alternatively, two or more of the blocks of methods 4200 may be performed in parallel.

A number of implementations have been described. Nevertheless, it will be understood that various modifications may be made without departing from the spirit and scope of the disclosure. Accordingly, other implementations are within the scope of the following claims.

These methods may work together to facilitate the assembly, operation, and connection of the medical device handle assembly, allowing for precise control and efficient functionality of the medical device.

### EXAMPLE CLAUSES

Example Clause 1: A medical device handle assembly comprising: a handle shell; a bevel gear assembly configured to be inserted into the handle shell, the bevel gear assembly comprising: a first bevel gear; and a second bevel gear in mechanical communication with the first bevel gear; wherein one end of the bevel gear assembly is configured to connect to an outer shaft knob; and wherein the second bevel gear includes a mating interface configured to engage with a servo shaft when the handle assembly is positioned on a stabilizer.

Example Clause 2: The medical device handle assembly of Example Clause 1, wherein the bevel gear assembly is configured to enable independent tip rotation of a medical device.

Example Clause 3: The medical device handle assembly of Example Clause 1 or Example Clause 2, wherein the bevel gear assembly may be removably inserted into the handle shell.

Example Clause 4: The medical device handle assembly of any one of Example Clauses 1-3, wherein the servo shaft is part of a servo-driven mechanism configured to provide precise control of tip rotation.

Example Clause 5: The medical device handle assembly of any one of Example Clauses 1-4, wherein the mating interface includes a safety feature configured to allow slipping when a predetermined force threshold is exceeded.

Example Clause 6: The medical device handle assembly of any one of Example Clauses 1-5, wherein the bevel gear assembly is configured to transmit rotational motion from the servo shaft to the outer shaft knob.

Example Clause 7: A method of operating a medical device handle assembly, the method comprising: inserting a bevel gear assembly into a handle shell; positioning the handle assembly on a stabilizer having a servo shaft; engaging a mating interface of the bevel gear assembly with the servo shaft; and actuating the servo shaft to rotate an outer shaft knob through the bevel gear assembly.

Example Clause 8: A medical device handle assembly comprising: a handle shell; a subassembly configured to slide into the handle shell, the subassembly comprising: pull lines configured for catheter deflection control; and an independent tip rotation mechanism; wherein the subassembly includes two spindles, a first spindle controlling left and right deflection and a second spindle controlling up and down deflection; and wherein the subassembly includes a mounting feature configured to accommodate bevel gears for enabling the independent tip rotation mechanism.

Example Clause 9: The medical device handle assembly of Example Clause 8, wherein the subassembly is pre-assembled as a single unit prior to insertion into the handle shell.

Example Clause 10: The medical device handle assembly of Example Clause 8 or Example Clause 9, wherein the handle assembly includes water-tight seals configured to provide durability and functionality.

Example Clause 11: The medical device handle assembly of any one of Example Clauses 8-10, wherein the pull lines and the independent tip rotation mechanism are integrated within a single cartridge structure.

Example Clause 12: The medical device handle assembly of any one of Example Clauses 8-11, wherein the mounting feature comprises a cartridge configured to receive the bevel gears.

Example Clause 13: The medical device handle assembly of any one of Example Clauses 8-12, wherein the first spindle and the second spindle are positioned to provide orthogonal deflection control.

Example Clause 14: The medical device handle assembly of any one of Example Clauses 8-13, wherein the subassembly may be easily removed from the handle shell for maintenance or replacement.

Example Clause 15: A method of assembling a medical device handle, the method comprising: pre-assembling pull lines and an independent tip rotation mechanism into a single subassembly; configuring the subassembly with two spindles for deflection control; and sliding the subassembly into a handle shell to form a complete handle assembly.

Example Clause 16: A motor adapter system for connecting a medical device handle to a motor, the system comprising: a motor adapter configured to be positioned on a motor shaft; a deflection knob insert configured to mate with the motor adapter; wherein the motor adapter includes alignment features configured to facilitate connection between the deflection knob insert and the motor adapter when misalignment is present; and wherein the alignment features are configured to allow quick connection and disconnection of the medical device handle in various operational conditions.

Example Clause 17: The motor adapter system of Example Clause 16, wherein the alignment features comprise tapered splines configured to guide the deflection knob insert onto the motor adapter.

Example Clause 18: The motor adapter system of Example Clause 16 or Example Clause 17, wherein the tapered splines are configured to allow power transmission while facilitating easier sliding engagement.

Example Clause 19: The motor adapter system of any one of Example Clauses 16-18, wherein the alignment features comprise: an intermediate adapter having a first set of teeth configured to engage with a second set of teeth on the motor adapter; a spring element configured to bias the intermediate adapter away from the motor adapter; and splined interfaces with chamfered features configured to facilitate alignment.

Example Clause 20: The motor adapter system of any one of Example Clauses 16-19, wherein the first set of teeth and the second set of teeth form a Hirth joint configured for self-alignment and positive engagement.

Example Clause 21: The motor adapter system of any one of Example Clauses 16-20, wherein the spring element is configured to be overcome by a force applied by the medical device handle to engage the intermediate adapter with the motor adapter.

Example Clause 22: The motor adapter system of any one of Example Clauses 16-21, wherein the motor adapter is configured to accommodate the misalignment that may occur when a user disconnects the medical device handle, uses the medical device handle manually, and then attempts to reconnect the medical device handle to the motor.

Example Clause 23: A method of connecting a medical device handle to a motor system, the method comprising: positioning a motor adapter on a motor shaft; aligning a deflection knob insert of the medical device handle with the motor adapter; engaging alignment features of the motor adapter to facilitate connection despite potential misalignment; and establishing mechanical coupling between the motor shaft and the deflection knob insert through the motor adapter.

Example Clause 24: The method of Example Clause 23, wherein engaging the alignment features comprises sliding the deflection knob insert onto tapered splines of the motor adapter.

Example Clause 25: The method of Example Clause 23 or Example Clause 24, wherein engaging the alignment features comprises: overcoming a spring force to engage complementary teeth of an intermediate adapter with teeth of the motor adapter; and aligning splined interfaces through chamfered features.

The foregoing disclosure provides illustration and description but is not intended to be exhaustive or to limit the implementations to the precise form disclosed. Modifications may be made in light of the above disclosure or may be acquired from practice of the implementations. As used herein, satisfying a threshold may, depending on the context, refer to a value being greater than the threshold, greater than or equal to the threshold, less than the threshold, less than or equal to the threshold, equal to the threshold, and/or the like, depending on the context. Although particular combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A medical device handle assembly comprising:
a handle shell;
a bevel gear assembly configured to be inserted into the handle shell, the bevel gear assembly comprising:
a first bevel gear; and
a second bevel gear in mechanical communication with the first bevel gear;
wherein one end of the bevel gear assembly is configured to connect to an outer shaft knob; and
wherein the second bevel gear includes a mating interface configured to engage with a servo shaft when the handle assembly is positioned on a stabilizer, wherein the stabilizer is configured control movement of the outer shaft knob via the servo shaft when the handle assembly is positioned on the stabilizer.

2. The medical device handle assembly of claim 1, wherein the bevel gear assembly is configured to enable tip rotation of a medical device.

3. The medical device handle assembly of claim 1, wherein the bevel gear assembly may be removably inserted into the handle shell.

4. The medical device handle assembly of claim 1, wherein the servo shaft is part of a servo-driven mechanism configured to provide precise control of tip rotation.

5. The medical device handle assembly of claim 1, wherein the mating interface includes a safety feature configured to allow slipping when a predetermined force threshold is exceeded.

6. The medical device handle assembly of claim 1, wherein the bevel gear assembly is configured to transmit rotational motion from the servo shaft to the outer shaft knob.

7. A medical device handle assembly comprising:
a handle shell;
a subassembly configured to be disposed in the handle shell, the subassembly comprising:
one or more pull lines configured for deflection control;
a tip rotation mechanism in communication with one or more bevel gears, wherein rotation of the one or more bevel gear causes rotation of the tip rotation mechanism; and
at least two spindles, a first spindle controlling deflection along a first axis and a second spindle controlling deflection along a second axis different from the first axis.

8. The medical device handle assembly of claim 7, wherein the subassembly is pre-assembled as a single unit prior to disposition into the handle shell.

9. The medical device handle assembly of claim 7, wherein the handle assembly includes water-tight seals configured to provide durability and functionality.

10. The medical device handle assembly of claim 7, wherein the pull lines and the tip rotation mechanism are integrated within a single cartridge structure.

11. The medical device handle assembly of claim 7, wherein the subassembly comprises a cartridge configured to receive the one or more bevel gears.

12. The medical device handle assembly of claim 7, wherein the first axis is orthogonal the second axis.

13. The medical device handle assembly of claim 7, wherein the subassembly is configured to be removeable from the handle shell for maintenance or replacement.

14. The medical device handle assembly of claim 7, wherein the at least two spindles are configured to be disposed on at least two servo shafts of a motorized stabilizer, wherein actuation of a first of the at least of the at least two servo shafts causes actuation of a first of the at least two spindles, and wherein actuation of a second of the at least two servo shafts causes actuation of a second of the at least two spindles.

15. A motor adapter system for connecting a medical device handle to a motor, the system comprising:
a motor adapter configured to be positioned on a motor shaft;
a deflection knob insert configured to mate with the motor adapter; and
wherein the motor adapter comprises at least one alignment component configured to facilitate connection between the deflection knob insert and the motor adapter and configured to prevent a connection between the deflection knob insert and the motor adapter when a misalignment is present.

16. The motor adapter system of claim 15, wherein the at least one alignment component comprises tapered splines configured to guide the deflection knob insert onto the motor adapter.

17. The motor adapter system of claim 16, wherein the tapered splines are configured to allow power transmission while facilitating easier engagement.

18. The motor adapter system of claim 15, wherein the at least one alignment component comprises:
an intermediate adapter having a first set of teeth configured to engage with a second set of teeth on the motor adapter;
a spring element configured to bias the intermediate adapter away from the motor adapter; and
splined interfaces with chamfered features configured to facilitate alignment.

19. The motor adapter system of claim 18, wherein the first set of teeth and the second set of teeth form a Hirth joint configured for self-alignment and positive engagement.

20. The motor adapter system of claim 18, wherein the spring element is configured to be overcome by a force applied by the medical device handle to engage the intermediate adapter with the motor adapter.

21. The motor adapter system of claim 15, wherein the motor adapter is configured to accommodate a usage misalignment that may occur when a user disconnects the medical device handle, uses the medical device handle manually, and then attempts to reconnect the medical device handle to the motor.
